(19) United States
(12) Patent Application Publication
(10) Pub. No.: US 2021/0002641 A1
(43) Pub. Date: Jan. 7, 2021

(54) COMPOSITIONS AND METHODS FOR TREATING GRAVES DISEASE

(71) Applicant: The Regents of the University of Michigan, Ann Arbor, MI (US)

(72) Inventors: Tae-Hwa Chun, Ann Arbor, MI (US); Fumihito Hikage, Ann Arbor, MI (US)

(21) Appl. No.: 16/981,181

(22) PCT Filed: Mar. 14, 2019
(86) PCT No.: PCT/US2019/022211

§ 371 Date: Sep. 15, 2020

Related U.S. Application Data
(60) Provisional application No. 62/643,822, filed on Mar. 16, 2018.

(51) Int. Cl. C12N 15/113; A61K 31/275; A61P 27/02

(52) U.S. Cl. CPC C12N 15/113; A61K 31/275; C12N 2310/14; C12N 15/1137; A61P 27/02

(57) ABSTRACT

Provided herein are compositions and methods for treating or preventing thyroid eye disease (e.g., related to Graves' disease). In particular, provided herein are compositions and methods for inhibiting or reducing the expression of HIF2A, LOX, or pathway components thereof.

This application claims the benefit of U.S. provisional application Ser. No. 62/643,822, filed Mar. 16, 2018, which is incorporated herein by reference in its entirety.

### STATEMENT REGARDING FEDERALLY SPONSORED RESEARCH OR DEVELOPMENT

This invention was made with government support under DK095137 awarded by the National Institutes of Health. The government has certain rights in the invention.

### FIELD

Provided herein are compositions and methods for treating or preventing thyroid eye disease (e.g., related to Graves' disease). In particular, provided herein are compositions and methods for inhibiting or reducing the expression of HIF2A, LOX, or pathway components thereof.

### BACKGROUND

Thyroid eye disease (TED) is an eye condition in which the inflammation and fibrosis of fat tissues and muscles around the eyes cause irritation, swelling, bulging eye, double vision, and even blindness. TED, also called as Graves' orbitopathy (GO), is most often associated with autoimmune hyperthyroidism (Graves' disease) and its prevalence is about 0.3% in population.

Activating autoantibody binding to the thyrotropin receptor (TSHR) underlies Graves' hyperthyroidism but the role of this molecular pathway in GO is less certain. Inter-species differences have confounded attempts to elucidate human diseases such as GO in mouse models (3, 4). Immunization of female BALB/c mice with cDNA encoding human TSHR-A subunit has yielded a phenotype resembling in some ways GO (5, 6), but similar studies have failed to reproduce Graves' ocular manifestations (7-9).

No effective pharmacological treatment for GO has been established to this date; therefore, patients with progressive TED need to undergo decompression surgeries.

Thus, pharmacological treatments for GO are needed.

### SUMMARY

Provided herein are compositions and methods for treating or preventing thyroid eye disease (e.g., related to Graves' disease). In particular, provided herein are compositions and methods for inhibiting or reducing the expression of HIF2A, LOX, or pathway components thereof.

Experiments described herein identified the HIF2A pathway as a target for treating and preventing thyroid eye disease. The compositions and methods described herein provide much needed pharmacological treatments for thyroid eye disease. Further experiments describe organoid disease modeling for TED using three-dimensional organoid culture derived from human orbital fibroblasts (Hikage et al. Endocrinology 2019: 1 (1): 20-35). Using this system, a biological pathway responsible for the pathogenesis of GO or TED was identified. The 3D disease modeling systems find use, for example, to conduct drug screening for TED or GO in vitro (e.g., to identify agents useful in treating or preventing thyroid eye disease).

For example, in some embodiments, provided herein is a method of preventing or treating thyroid eye disease in a subject, comprising: inhibiting at least one activity or downregulating the expression of hypoxia-inducible factor alpha (HIF2A) or Lysyl Oxidase (LOX) in the subject under conditions such that the thyroid eye disease is treated or prevented. In some embodiments, the inhibiting or downregulating the expression of HIF2A or LOX comprises the use of an agent selected from, for example, a nucleic acid, a small molecule, a peptide, a nucleic acid-containing vector, or an antibody. The present disclosure is not limited to particular nucleic acids. Examples include, but are not limited to, a siRNA, miRNA, an antisense nucleic acid, or an shRNA (e.g., delivered using a lentiviral vector). The present disclosure is not limited to particular small molecules. Examples include, but are not limited to, PT2385, R-aminopropionitrile (BAPN), CH₁₂H₆ClFN₄O₃, PT2385, or PT2399. In some embodiments, the inhibiting comprises inhibiting at least one activity or altering the expression of a HIF2A or LOX pathway member. In some embodiments, the subject has Graves' disease.

Additional embodiments provide a method of altering HIF2A or LOX activity in a cell, comprising: inhibiting at least one activity or downregulating the expression of HIF2A or LOX in the cell. In some embodiments, the cell is in vitro, ex vivo, or in vivo (e.g., in a subject).

Yet other embodiments provide an agent that inhibits at least one activity or downregulates the expression of HIF2A or LOX for use in treating or preventing thyroid eye disease in a subject.

Still other embodiments provide the use of an agent that inhibits at least one activity or downregulates the expression of HIF2A or LOX for treating or preventing thyroid eye disease in a subject.

Further embodiments provide methods and uses of inhibiting HIF2A and/or LOX for use in drug screening, research (e.g., to characterize disease), etc.

Additional embodiments are described herein.

### DESCRIPTION OF THE FIGURES

FIG. 1 shows that 3D OF organoids recapitulate GO tissue stiffness. (a) OFs isolated from surgical waste samples of non-GO (N-OF) and GO subjects (G-OF) cultured under conventional 2D culture conditions. (b) Schematic protocols for non-adipogenic and adipogenic culture of 3D OF organoids. (c) Representative micrographs of 3D organoids formed by N-OFs and GOFs cultured with and without adipogenic cocktail (−, + adipogenesis). (d) Organoid cross-sectional area (CSA) over the culture time course, with adipogenic mix (+Adip.) and without adipogenic mix. (e) Microindentation-based measurement of tissue stiffness measured after 6 and 12 days culture in standard medium (non adipogenic condition). (f) The effect of adipogenesis on tissue stiffness.

FIG. 2 shows that ECM deposition determines the tissue stiffness of 3D OF organoids. (a) Representative Sirius red-stained sections. (b) Representative immunofluorescent staining of collagen family members and FN (green) with DAPI (blue). (c) Gene expression of collagen family members and FN. n=3-5 independent experiments. (d) Representative immunofluorescent staining of collagen family members and FN (green) and nucleus (DAPI, blue) in 3D G-OF organoids, which were treated with and without MMP inhibitor (GM6001). (e) Microindentation-based measurement of organoid tissue stiffness.

FIG. 3 shows that thyrotropin receptor stimulation increases ECM deposition and tissue stiffness of 3D G-OF organoids. (a) Experimental protocol. (b) Organoid size. n=15 organoids. (c) The effect of 30 nM triiodothyronine (T3), 5 mIU/ml thyrotropin (TSH), and combination on the tissue stiffness of 3D G-OF (n=12-16 organoids) and dose-dependent effect of TSH on 3D G-OF organoids. n=8-9. (d) The effects of TSH and T3 on the tissue stiffness of N-OF (n=11) organoids. (e) TSH-dependent accumulation of COL6 and FN. (f) The summarized effects of MMP inhibitor, adipogenesis (adip.) and TSH on the tissue stiffness of 3D N-OF and G-OF organoids. (g) Effects of M22 thyrotropin stimulating antibody (5 mg/mL) on G-OF and N—OF organoid stiffness. n=10 to 11

FIG. 4 shows that Lysyl oxidase (LOX) regulates the tissue stiffness of 3D G-OF organoids. (a) Increased expression of LOX and CTGF in G-OFs under 3D but not 2D culture conditions. (b) Immunofluorescent staining of LOX and CTGF in 3D OF organoids. (c) TSH-dependent induction of LOX in 3D G-OF organoids. (d) Effect of LOX inhibitor, BAPN, on ECM accumulation in 3D G-OF organoids. (e) Effect of BAPN on the tissue stiffness of 3D G-OF organoids (control and TSH-stimulated).

FIG. 5 shows inflammatory characteristics of 3D G-OF organoids. (a) Real-time qPCR of inflammatory genes in 3D OF organoids formed by N- and G-OFs. (b) Real-time qPCR of IL B in 2D culture condition of N- and G-OFs. (c) Real-time qPCR of IL-1B and IL6 in 3D OF organoids treated with T3, TSH and combination. (d) Representative micrographs demonstrating GFP-labeled fibrocyte invasion of 3D organoids derived from N-OFs and G-OFs. (e) Real-time qPCR of inflammatory genes in 3D OF organoids formed by N-OFs and G-OFs co-cultured with 2,000 cells of fibrocytes (N+F and G+F) as well as the expected contribution of 10% fibrocyte alone (F).

FIG. 6 shows that HIF2A contributes to LOX induction and tissue stiffness. (a) 3D-specific elevation of HIF2A expression in G-OFs. (b) Immunofluorescent staining of HIF2A and HIF1A within N-OF and G-OF 3D organoids. HIF2A, HIF1A (green) and DAPI (blue). (c) TSH-dependent induction of HIF2A within 3D G-OF organoids detected by immunofluorescent staining. (d) Downregulation of HIF2A target genes in G-OF organoids treated with shRNA against HIF2A. (e) Effects of shRNA-mediated HIF2A suppression on HIF2A dependent accumulation of LOX. (f) Effects of shRNA-mediated HIF2A suppression on tissue stiffness; three-independent shRNA clones examined against vehicle and empty lentivirus-treated groups. (g) Suppression of HIF2A-LOX-dependent accumulation of COL1, COL4, COL6, and FN. (h) The effect of small molecular HIF2A antagonist (compound 2) on LOX and FN expression in 3D G-OF organoids.

FIG. 7 shows that expression of oxygen-resistant HIF2A is sufficient to induce LOX and tissue stiffness in mouse OFs. (a) Isolation of mouse orbital fibroblasts (upper), generation of 3D mouse OF organoids treated with control and Cre-expressing adenoviruses (lower). (b) Real-time qPCR of HIF2dPA and LOX in 3D mouse OF organoids. (c) HIF2A-dependent induction of LOX. (d) HIF2A-dependent regulation of COL1, COL4, COL6, and FN contents within organoids. (e) Cre-dependent induction of mutant HIF2A caused increased tissue stiffness of mouse OF organoids. (f) Real-time qPCR of IL1B and IL6 in 3D mouse OF organoids.

FIG. 8 shows pathological expression of HIF2A and LOX in GO tissues. (a) Over-representation of HIF2A and LOX and the accumulation of COL6 and FN in GO tissues. Non-GO (n=5) and GO (n=10) tissue slides examined for the expression of HIF2A (green) and LOX (red). (b) Positive correlation between expression levels of HIF2A and LOX. (c) Representative confocal image of HIF1A in human orbital tissues. HIF1A (green) and DAPI (blue).

FIG. 9 shows viability of organoids formed by N- and G-OFs.

FIG. 10 shows ECM deposition in conventional 2D culture condition.

FIG. 11 shows inflammatory gene expression of 3D fibrocytes organoids.

FIG. 12 shows that knockdown of HIF1A in 3D organoid does not affect the gene expression of LOX and CTGF (a) HIF1A shRNA effect on tissue stiffness. (b) Real-time qPCR of genes in 3D G-OF organoids.

FIG. 13 shows the effect of PT2385 on inhibition of HIF2-dependent induction LOX activity.

FIG. 14 shows that an allosteric HIF2A antagonist reverses TSH- and M22-dependent tissue stiffness.

### DEFINITIONS

To facilitate an understanding of the present disclosure, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., thyroid eye disease). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of an agent (e.g., an agent described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., agents described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “toxic” refers to any detrimental or harmful effects on a cell or tissue as compared to the same cell or tissue prior to the administration of the toxicant.

### DETAILED DESCRIPTION OF THE DISCLOSURE

Provided herein are compositions and methods for treating or preventing thyroid eye disease (e.g., related to Graves' disease). In particular, provided herein are compositions and methods for inhibiting or reducing the expression of HIF2A, LOX, or pathway components thereof.

Three-dimensional (3D) tissue culture is effective to recapitulate in vivo tissue microenvironments for disease modeling and drug discovery. Described herein is the development of a high-throughput 3D organoid culture system for orbital adipose tissue-derived fibroblasts. This system allowed for modeling pathological 3D tissue stiffness, ECM remodeling, and inflammatory gene expression observed in GO. Experiments described herein demonstrated that hypoxia inducible factor-2 alpha (HIF2A) accelerates ECM deposition in GO through the induction of lysyl oxidase (LOX). Suppressing HIF2A (e.g., by shRNA) or blocking HIF2A or LOX activity by chemical antagonist effectively ameliorated fibrotic tissue remodeling in GO organoids. Conversely, the overexpression of HIF2A was sufficient to induce fibrotic tissue remodeling and stiffness in 3D organoids. Validating the findings obtained through 3D disease modeling, HIF2A and LOX were highly upregulated in tandem within human GO tissues.

Accordingly, provided herein are compositions and methods for treating and preventing thyroid eye disease by inhibiting HIF2A, LOX, or pathway components thereof. Exemplary compositions and methods are described herein.

### I. Inhibitors

In some embodiments, the HIF2A and/or LOX inhibitor is selected from, for example, a nucleic acid (e.g., siRNA, shRNA, miRNA or an antisense nucleic acid), a small molecule, a peptide, or an antibody.

Exemplary small molecule inhibitors include, but are not limited to, β-aminopropionitrile (BAPN), C₁₂H₆ClFN₄O₃, PT2385

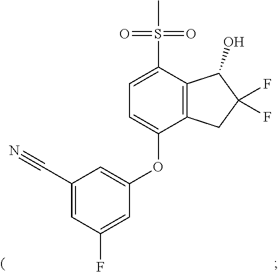 [SMILES: CS(=O)(=O)c1ccc(Oc2cc(F)cc(C#N)c2)c2c1[C@H](O)C(F)(F)C2]

Courtney et al., Journal of Clinical Oncology—published online before print Dec. 19, 2017; herein incorporated by reference in its entirety), PT2399

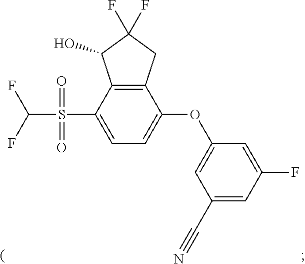 [SMILES: N#Cc1cc(F)cc(Oc2ccc(S(=O)(=O)C(F)F)c3c2CC(F)(F)[C@H]3O)c1]

Chen et al., Nature. 2016 Nov. 3; 539(7627):112-117: herein incorporated by reference in its entirety),

PT2385

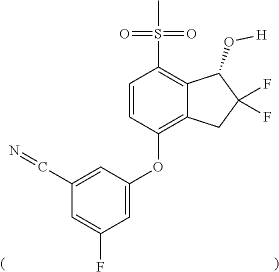 [SMILES: CS(=O)(=O)c1ccc(Oc2cc(F)cc(C=N)c2)c2c1[C@H](O)C(F)(F)C2]

(Peleton Therapeutics, Dallas, Tex.) and those described in Martinez-Saez et al., Critical Reviews in Oncology/Hematology, 111 (2017) 117 and U.S. Pat. Nos. 9,796,697, 9,884,843, 9,896,418, and 9,908,845; each of which is herein incorporated by reference in its entirety.

In some embodiments, the HIF2A and/or LOX inhibitor is a nucleic acid. Exemplary nucleic acids suitable for inhibiting HIF2A and/or LOX (e.g., by preventing expression of HIF2A and/or LOX) include, but are not limited to, antisense nucleic acids and RNAi nucleic acids. In some embodiments, nucleic acid therapies are complementary to and hybridize to at least a portion (e.g., at least 5, 8, 10, 11, 12, 13, 14, 15, 16, 17, 18, 19, or 20 nucleotides) of HIF2A (e.g., as described by Accession No. NM_001430.4) and/or LOX (e.g., as described by Accession No. NM_002317.6).

In some embodiments, compositions comprising oligomeric antisense compounds, particularly oligonucleotides are used to modulate the function of nucleic acid molecules encoding HIF2A and/or LOX, ultimately modulating the amount of HIF2A and/or LOX expressed. This is accomplished by providing antisense compounds that specifically hybridize with one or more nucleic acids encoding HIF2A and/or LOX. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is decreasing the amount of HIF2A and/or LOX proteins in the T-cell.

In some embodiments, nucleic acids are RNAi nucleic acids. “RNA interference (RNAi)” is the process of sequence-specific, post-transcriptional gene silencing initiated by a small interfering RNA (siRNA), shRNA, or microRNA (miRNA). During RNAi, the RNA induces degradation of target mRNA with consequent sequence-specific inhibition of gene expression.

In “RNA interference,” or “RNAi,” a “small interfering RNA” or “short interfering RNA” or “siRNA” or “short hairpin RNA” or “shRNA” molecule, or “miRNA” an RNAi (e.g., single strand, duplex, or hairpin) of nucleotides is targeted to a nucleic acid sequence of interest, for example, HIF2A and/or LOX.

An “RNA duplex” refers to the structure formed by the complementary pairing between two regions of a RNA molecule. The RNA using in RNAi is “targeted” to a gene in that the nucleotide sequence of the duplex portion of the RNAi is complementary to a nucleotide sequence of the targeted gene. In certain embodiments, the RNAi is are targeted to the sequence encoding HIF2A and/or LOX. In some embodiments, the length of the RNAi is less than 30 base pairs. In some embodiments, the RNA can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the RNAi is 19 to 32 base pairs in length. In certain embodiment, the length of the RNAi is 19 or 21 base pairs in length.

In some embodiments, RNAi comprises a hairpin structure (e.g., shRNA). In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase.

As used herein, Dicer-substrate RNAs (DsiRNAs) are chemically synthesized asymmetric 25-mer/27-mer duplex RNAs that have increased potency in RNA interference compared to traditional RNAi. Traditional 21-mer RNAi molecules are designed to mimic Dicer products and therefore bypass interaction with the enzyme Dicer. Dicer has been recently shown to be a component of RISC and involved with entry of the RNAi into RISC. Dicer-substrate RNAi molecules are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional RNAi molecules. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs.

“Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (e.g., about 35 nucleotides upstream and about 40 nucleotides downstream) arise from restriction enzyme sites within the multiple cloning site of the RNAi. As used herein the term “miRNA” encompasses both the naturally occurring miRNA sequences as well as artificially generated miRNA shuttle vectors.

The RNAi can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

The present disclosure contemplates the use of any genetic manipulation for use in modulating the expression of HIF2A and/or LOX. Examples of genetic manipulation include, but are not limited to, gene knockout (e.g., removing the HIF2A and/or LOX gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, lentiviral vectors, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

In some embodiments, vectors are lentiviral vectors. Lentiviruses are a subclass of retroviruses. They are sometimes used as vectors for gene therapy thanks to their ability to integrate into the genome of non-dividing cells, which is the unique feature of lentiviruses as other Retroviruses can infect only dividing cells. The viral genome in the form of RNA is reverse-transcribed when the virus enters the cell to produce DNA, which is then inserted into the genome at a random position by the viral integrase enzyme.

For safety reasons lentiviral vectors do not carry the genes required for their replication. To produce a lentivirus, several plasmids are transfected into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

Vectors may be administered to subject in a variety of ways. For example, in some embodiments of the present disclosure, vectors are administered into tumors or tissue associated with tumors using direct injection. In other embodiments, administration is via the blood or lymphatic circulation (See e.g., PCT publication 1999/02685 herein incorporated by reference in its entirety). Exemplary dose levels of adenoviral vector are preferably 10⁸ to 10¹¹ vector particles added to the perfusate.

In some embodiments, nucleic acids (e.g., nucleic acids that inhibit the expression of HIF2A and/or LOX) are introduced into the genome using the RNA-guided microbial endonuclease CRISPR (clustered regularly interspaced short palindromic repeat)/Cas9 (CRISPR associated protein 9) system. The CRISPR/Cas9 system allows precise genome editing. It is widely used for studying the functionality of genetic elements, creating genetically modified organisms, and is promising in clinical therapeutic applications. Cas9 is an RNA-guided nuclease that catalyzes site-specific cleavage of double stranded DNA. A guide RNA comprising a 20-nt seed region complementary to its target activates Cas9 nuclease and creates a DNA double strand break (DSB).

The CRISP/CAS9 system can be used for sequence-specific gene editing and transcriptional regulation (Cho et al., 2013 Nat. Biotechnol. 31, 230-232; Cong et al., 2013 Science 339, 819-823; Fu et al., 2014 Nat. Biotechnol. 32, 279-284; Jinek et al. Science 337, 816-821, 2012; Mali et al., 2013b Science 339, 823-826; Qi et al., 2013 Cell 152, 1173-1183; Ran et al., 2015 Nature 520, 186-191; Yu et al., 2015 Cell Stem Cell 16, 142-147).

In some embodiments, the present disclosure provides antibodies that inhibit HIF2A and/or LOX. Any suitable antibody (e.g., monoclonal, polyclonal, or synthetic) may be utilized in the therapeutic methods disclosed herein. In some embodiments, the antibodies are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, candidate HIF2A and/or LOX inhibitors are screened for activity (e.g., using the methods described herein or another suitable assay).

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### II. Methods of Preventing or Treating Thyroid Eye Disease

Provided herein are methods of treating or preventing thyroid eye disease (e.g., associated with Graves' disease) through inhibition of HIF2A and/or LOX.

In some embodiments, the compounds and pharmaceutical compositions described herein are administered in combination with one or more additional agents, treatment, or interventions (e.g., agents, treatments, or interventions useful in the treatment of thyroid eye disease or Graves' disease).

In some embodiments, HIF2A and/or LOX inhibitors are co-administered with an existing treatment for thyroid eye disease (e.g., decompression therapy) or Graves' disease (e.g., radioactive iodine or propylthiouracil).

In some embodiments, therapies described herein are administered to subjects diagnosed with Graves' disease or overactive thyroid of other causes but not exhibiting signs or symptoms of thyroid eye disease (e.g., in order to prevent development of thyroid eye disease).

In some embodiments, subjects are monitored during treatment for signs or symptoms of thyroid eye disease or expression of HIF2A and/or LOX. In some embodiments, treatments are modified (e.g., increased, changed, or decreased) based on the signs or symptoms of thyroid eye disease or expression of HIF2A and/or LOX.

### EXPERIMENTAL

The following examples are provided in order to demonstrate and further illustrate certain preferred embodiments and aspects of the present disclosure and are not to be construed as limiting the scope thereof.

### Example 1

### Methods

### Materials

Dulbecco's Modified Eagle's Medium (DMEM) (#11965092, GibcoThermo Fisher Scientific. Waltham, Mass.), fetal bovine serum (FBS) (#16-000-044, GibcoThermo Fisher Scientific), Lglutamin (#25030081, GibcoThermo Fisher Scientific), antibiotic/antimycotic (#15240062, Gibco/Thermo Fisher Scientific), penicillin/streptomycin (#15140122, Gibco/Thermo Fisher Scientific), Ficoll-Paque Plus (#17-1440-03, GE Healthcare, Piscataway, N.J.), Puromycin (#P8833. Sigma-Aldrich, St Louis, Mo.), Protamine sulfate salt from salmon (#P4020, Sigma-Aldrich), Methocel A4M (#94378, Sigma-Aldrich), Dexamethasone (#D1756, Sigma-Aldrich), 3,3′,5-Triiodo-L-thyronine (T3) (#T6397, Sigma-Aldrich), Troglitazone (#71750, Cayman Chemical, Ann Arbor, Mich.). Porcine insulin (#15523, Sigma-Aldrich), Thyrotropic hormone from bovine pituitary (TSH) (#T8931, Sigma-Aldrich), HIF-2 Antagonist 2 (#SML0883, Sigma-Aldrich), Insolution GM6001 (#364206, Calbiochem, San Diego, Calif.), 3-Aminopropionitrile fumarate salt (β-aminopropionitrile (BAPN), #A3134, Sigma-Aldrich), and M22 TSH-stimulating human monoclonal antibody (RSR Diagnostics for Autoimmunity, Cardiff, United Kingdom).

### Human OF Isolation and Culture

Orbital adipose tissues were obtained from surgical waste samples of de-identified euthyroid patients with GO undergoing orbital decompression and from non-GO subjects without inflammatory orbital disease, who underwent cosmetic eyelid surgery. OFs were isolated and grown as previously described (49). Briefly, tissues were minced into small pieces, placed on 150 mm culture dishes, and submerged in growth medium (DMEM supplemented with 10% v/v FBS, 1% v/v L-glutamine, 1% v/v antibiotic-antimycotic) at a sufficient depth to cover the tissue chunks. Explants were cultured in a humidified incubator
(at 37° C. with 5% CO₂), with growth medium changes every 2-3 days. OFs from five GO patients five non-GO patients were isolated and expanded for subsequent experiments. All experiments were performed using OFs of 3-6 passages after the initial cell isolation.

### Mouse OF Culture

ROSA26-STOP-HIF2dPA (flox/flox) mice were provided by Dr. Emestina Schipani (University of Michigan), and used for the isolation of orbital adipose tissue. Cells isolated from this depot were cultivated as previously reported (50) and cultured in a standard growth medium. A degradation-resistant HIF2A mutant was introduced by adenoviral Cre (Vector; Ad5 CMV-Cre)-recombinase by incubation for 16 hours with the virus. Adenoviral GFP (Vector; Ad5 CMV eGFP.dIE3) or Empty (Vector: Ad5 CMV pLpA.dIE3)-recombinase was used as controls.

Fibrocyte Differentiation from Monocytes

Fibrocytes were isolated from peripheral blood mononuclear cells (PBMCs) as previously described (51). Briefly, Red Cross Filters (ATS LPL, Pall Medical, East Hills, N.Y.) used for PBMC removal were obtained from the Blood Bank. After extraction from the filter with PBS wash, cells were transferred into a 50-mL conical tube containing 10 ml of Ficoll-Paque Plus. Cells were then centrifuged for 25 minutes at 1,100 g. PBMCs were collected and centrifuged for 10 minutes at 1,100 g and the pellet was resuspended in PBS. After centrifugation for 6 min at 1,100 g, the pellet was resuspended in DMEM containing 10% v/v FBS, 1% v/v glutamine, 1% v/v penicillin/streptomycin. PBMCs were seeded at a density of 5×10⁶ cells in each well of a 6-well plate. Unattached cells were discarded seven days later, and adherent monolayers were cultured for an additional 3 to 5 days until used for experiments. For co-culture of OFs and fibrocytes, OF organoids were generated by suspending 20,000 OFs in a 25 μL drop of standard medium with methylcelluose in a drop culture plate (as described below). 2,000 fibrocytes were added into each droplet on day 1 after OF seeding. For GFP-based cell labeling, fibrocytes were treated with adenoviral GFP (Vector; Ad5 CMV eGFP.dlE3) for 16 hours as described above.

### Three-Dimensional Culture of Organoids

A hanging droplet spheroid culture system was used to generate 3D organoids. To facilitate stable morphology, methylcellulose (Mehocel A4M) was added to the growth medium (52). Prior to seeding the hanging drop culture plate (#HDP1385, Sigma-Aldrich), cells were cultured in 100 mm or 150 mm dishes until reaching approximately 90% confluence. After washing with Hank's Balanced Salt Solution (HBSS), cells were detached using 0.25% Trypsin/EDTA and resuspended in growth medium. After centrifugation for 5 minutes at 300 g, the cell pellet was re-suspended in growth medium containing 0.25% w/v Methocel A4M. Volume was adjusted such that 20,000 cells were contained in 25 μL solution, and 25 μL drops were placed into each well of the drop culture plate (defined as day 0). Organoid medium (growth medium with 0.25% w/v methocel A4M) was used throughout the duration of spheroid culture. Every day, 14 μL culture medium was removed and 14 μL fresh culture medium added to each well.

Adipogenic Differentiation of Organoids and Treatment with Inhibitors

Adipogenic differentiation was induced with (growth or organoid) medium containing 250 nM dexamethasone, 10 nM T3, 10 μM troglitazone, and 1 μg/ml insulin on days 1-5, followed by medium with 10 μM troglitazone and 1 μg/ml insulin on days 6-11, then with medium alone on day 12. For the analysis of the lipid droplet formation, organoids were transferred to super-low attachment 6 well dishes and incubated in HBSS containing BODIPY (#D3922, Thermo Fisher Scientific) at 1:500 ratio by volume for 1 hour, then fixed in 4% paraformaldehyde (PFA) in PBS for 10 minutes at room temperature. Fluorescence intensity of BODIPY-stained lipid droplets were measured using Nikon A1 confocal microscope (Tokyo, Japan) and quantified using Image J software version 1.51n (NIH, Bethesda, Md.). Several compounds were added to droplets on day 1 and maintained at the same concentration until collection of organoids on day 6. For individual experiments, these included 10 μM GM6001, (global MMP inhibitor), 30 nM T3, 5 mIU/ml TSH, 30 nM T3+5 mIU/ml TSH, 0.5 mM BAPN (LOX inhibitor), and 1 μM, 5 μM or 10 μM HIF2A antagonist.

### Mechanical Testing (Compression Test)

The mechanical testing of the organoids was performed using the Microsquisher (CellScale, Waterloo, ON, Canada) as recently reported (53). The device consists of a microscale parallelplate compression system equipped with a 406-μm diameter cantilever. Organoids placed on a 3 mm-square plate for analysis. Immediately following collecting, organoids were placed in a 40 mL HBSS fluid bath at 37° C. and the cantilever was lowered until the upper compression plate was in contact with the top of the organoid. The organoids were compressed to 50% deformation (as determined by microscopic camera) for 20 seconds. The force used to produce 50% stain of displacement was measured by the cantilever and data were reported as force/displacement (μN/μm).

### Sirius Red Staining

For Sirius Red (SR) staining, sections of orbital adipose tissue were incubated in a solution consisting of 0.1% Direct Red 80 (#B21693, Alfa Aesar, Tewksbury, Mass.) in picric acid for 1 hour at room temperature with agitation. They were next transferred to a 0.5% glacial acetic acid solution and incubated for 10 minutes at room temperature with agitation. Sections were then washed briefly in tap water, dehydrated through an ethanol series, briefly incubated in xylene and cover-slipped with Permount (Thermo Fisher Scientific, Carlsbad, Calif.) for microscopic analysis.

### Imnunostaining of Organoids and Tissues

Immunofluorescent staining of HIF2A, HIF1A, LOX, COL6, and FN protein in orbital adipose tissues was performed on paraffin-embedded sections of orbital tissues from 5 non-GO subjects and 10 GO patients. Sections (7-μm thick) of paraffinized orbital adipose tissue were deparaffinized, rehydrated, and permeabilized with cold acetone for 30 seconds. After blocking with 5% normal goat serum/0.1% Triton X-100 in PBS (PBST) for 1 hour at room temperature, sections were incubated overnight at 4° C. with rabbit anti-HIF2A monoclonal antibody at 1:200 dilution (#A700-003, Bethyl laboratories Inc. Montgomery, Tex.), rabbit anti-HIF1A monoclonal antibody at 1:200 dilution (#A700-001, Bethyl laboratories Inc.), mouse anti-lysyl oxidase antibody (#sc-373995, 1:200, Santa Cruz Biotechnology, Santa Cruz, Calif.), rabbit anti-collagen VI antibody (#600-401-108-0.1, 1:200), or mouse anti-fibronectin antibody (#sc-8422, 1:200). After a subsequent wash in PBST, sections were incubated with goat Alexa Fluor 488 anti-rabbit IgG (#A-11070, 1:1000, Invitrogen, Carlsbad, Calif.) or goat Alexa Fluor 594 anti-mouse IgG (#A-11020, Invitrogen) for 1 hour at room temperature. Slides were counterstained with DAPI (#D1306, Invitrogen) and mounted in Prolong Gold Antifade Reagent (#P36931, Invitrogen).

Organoids were fixed in 4% PFA/PBS overnight with or without permeabilization in 0.5% Triton X-100 in PBS for 1 hour. To stain extracellular collagen and fibronectin, no permeabilization was performed, and all the detergents were excluded from the subsequent procedures. After blocking in 3% BSA/0.1% PBST for 3 hours at room temperature, organoids were washed 3 times for 30 minutes with PBST. Samples were incubated with primary antibody overnight at 4° C. The catalog numbers the dilution of primary antibodies were as follows: rabbit anti-HIF2A monoclonal antibody (#A700-003, 1:200), rabbit anti-HIF1A monoclonal antibody (#A700-001, 1:200), both from Bethyl laboratories Inc.: rabbit anti-collagen I antibody (#600-401-103-0.5, 1:200), rabbit anti-collagen III antibody (#600-401-105-0.1, 1:200), rabbit anti-collagen IV antibody (#600-401-106-0.5, 1:200), rabbit anti-collagen V antibody (#600-401-107-0.1, 1:200), rabbit anti-collagen VI antibody (#600-401-108-0.1, 1:200), all from Rockland Immunochemicals Inc.; rabbit anti-alpha smooth muscle actin antibody (#5694, 1:100), rabbit anti-Ki67 antibody (#15580, 1 μg/ml), from Abcam, Cambridge, UK; rabbit anti-cleaved caspase-3 antibody (#9664, 1:400) from Cell Signaling Technology, Inc. Danvers, Mass., mouse anti-fibronectin antibody (#sc-8422, 1:200), mouse anti-connective tissue growth factor antibody (#sc-365970, 1:200), mouse anti-lysyl oxidase antibody (#sc-373995, 1:200), mouse anti-Thy-1 antibody (#sc-19614, 1:200), all from Santa Cruz Biotechnology. After a subsequent wash in PBST, the organoids were incubated with goat Alexa Fluor 488 anti-rabbit IgG (#A-11070, 1:500, Invitrogen, Carlsbad, Calif.) and goat Alexa Fluor 594 anti-mouse IgG (#A-11020, 1:500, Invitrogen) for 3 hours at room temperature. Alexa Fluor 594 phalloidin (#A12381, Invitrogen) was used for F-actin staining and DAPI (#D1306, Invitrogen) for nuclear staining. Samples were mounted in Prolong Gold as indicated above.

For 2D cell culture, cells were grown using a 4-well chamber slide (#154526PK, Thermo Fisher Scientific) until 80-90% confluence and fixed with 4% PFA for 1 hour at room temperature. After repeated washes in PBST, cells were permeabilized with 0.3% Triton X-100 for 5 min. When staining extracellular collagen and fibronectin, permeabilization step was omitted and detergents were excluded in subsequent procedures. After blocking cells with 1% bovine serum albumin (BSA) for 1 hour at room temperature, primary antibody was incubated overnight at 4° C. After repeated washes, the samples were incubated with the secondary antibodies (1:1000) mentioned above for corresponding primary antibodies, Alexa Flour 594 Phalloidin (#A12381, Invitrogen) for F-actin, Cells were then incubated in DAPI for 1 hour at room temperature, before mounting with Prolong Gold Antifade Reagent (#P36931, Invitrogen).

### Image Acquisition and Analysis

Bright field images of each organoid were captured in 4× or 10× objective lenses using inverted microscope (Olympus IX70). The largest cross-sectional area (CSA) was calculated using Image J software version 1.51n. Immunofluorescent images were obtained using Nikon A1 confocal microscopy and NIS element 4.0 software (Tokyo, Japan). Images in 2D were acquired with a 20× air objective or 100× oil objective with a resolution of 1,024×1,024 or 2,048×2,048 pixels. For images of organoids, serial z-axis imaging (2.2 μm interval) at 65 μm range from a surface of organoids was conducted using a 20× air objective with a resolution of 512×512, 1,024×1,024, or 2,048×2,048 pixels and was converted as Z-stack image using the maximum intensity projection feature of NIS element 4.0 software. Intensity of immunofluorescent target proteins was quantified using NIH ImageJ software. Signal intensity of organoids was expressed as intensity/surface area measured at 65 μm from the top of the organoid in the z-plane. The surface area was calculated as follows: Surface area=D×A/(A+π×H²), where D (μm)=organoid diameter, A (μm2)=area of sectioned organoid, H (μm)=height=65 μm.

shRNA, Lentvruses, and Dansduedon

For HIF2A knockdown, lentiviruses carrying two unique HIF2A shRNA constructs in pLenti-GipZCMV-Puro (GE healthcare) or two unique pLenti-LKO.1-puro (Sigma-Aldrich) were transduced with 50 μg/ml Protamine for 16 hours. For HIF1A knockdown, lentivirus carrying two unique HIF1A shRNA in two of pLenti-LKO.1-puro vector (Sigma-Aldrich) were transduced with 50 μg/ml Protamine for 16 hours. shRNA sequences are as follows:

HIF2A knockdown #1, Lenti-GipZ-HIF2A-VSVG,
(SEQ ID NO: 1)
GCATTAAAGCAGCGTATC,
#2, Lenti-LKO-HIF2A-3805-VSVG,
(SEQ ID NO: 2)
CCGGGCGCAAATGTACCCAATGATACTCGAGTATCATTGGGTACATTTGCG
CTTTTT,
#3, Letni-LKO-HIF2A-3806-VSVG,
(SEQ ID NO: 3)
CCGGCAGTACCCAGACGGATTTCAACTCGAGTTGAAATCCGTCTGGGTACT
GTTTTT,
HIF1A knockdown #1, Letni-LKO-HIF1A-3810-VSVG,
(SEQ ID NO: 4)
CCGGGTGATGAAAGAATTACCGAATCTCGAGATTCGGTAATTCTTTCATCA
CTTTTT,
#2, Letni-LKO-HIF1A-10819-VSVG,
(SEQ ID NO: 5)
CCGGTGCTCTTTGTGGTTGGATCTACTCGAGTAGATCCAACCACAAAGAGC
ATTTTT.

After transduction, selection was performed using 1 μg/ml puromycin.

### Real-Time PCR

Total RNA was extracted from 20 organoids using RNeasy mini kit (Qiagen, Valencia, Calif.). Reverse transcription was performed with the SuperScript II kit (Invitrogen) as per manufacturer's instructions. Respective gene expression was quantified by real-time PCR with either Power SYBR green or Universal Taqman Master mix using a StepOnePlus machine (Applied Biosystems/Thermo Fisher scientific). cDNA quantities were normalized to the expression of housekeeping gene 3684 (Rplp0) and are shown as fold-change relative to control. Sequences of primers and Taqman probes used are shown in Table 1.

### Statistics

All statistical analyses were performed using Graph Pad Prism 7 (GraphPad Software, San Diego, Calif.). For comparison of two mean values, a two-tailed Student's t-test was used to calculate statistical significance with a confidence level greater than 95%. To analyze the difference in groups, a grouped analysis with two-way analysis of variance (ANOVA) followed by a Tukey's multiple comparison test was performed. Data are presented as arithmetic means±standard error of the mean (SEM).

### Results

### 3D Organolds Formed by G-OFs Mimic In Vivo-Like Tissue Remodeling and Stiffness

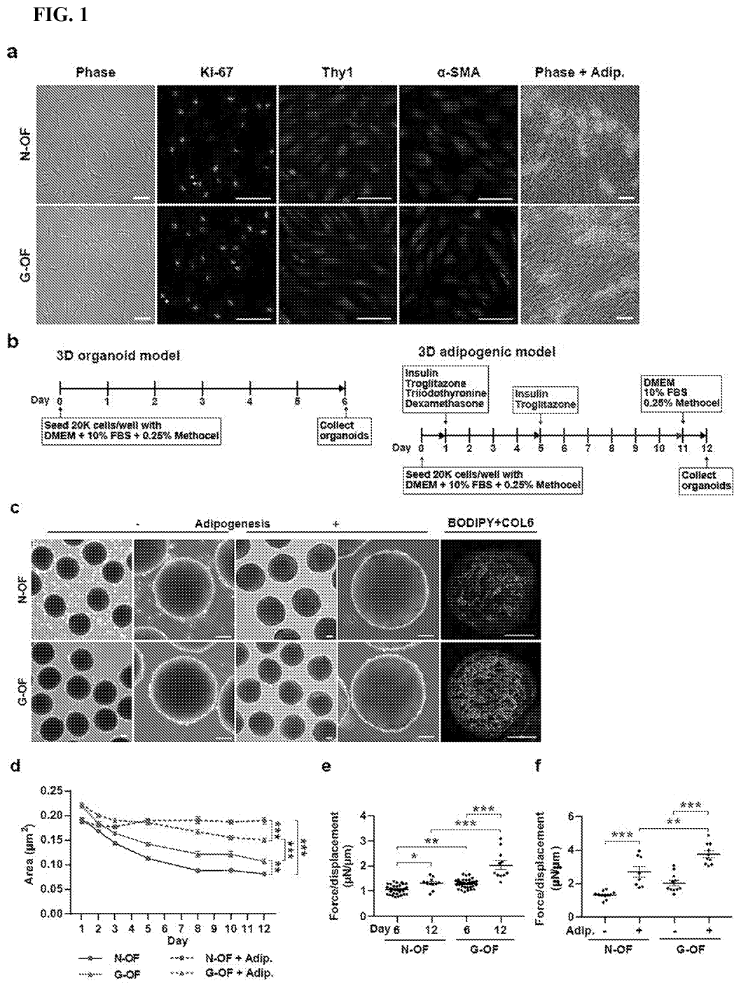

Primary OFs were isolated from de-identified surgical waste of nasal superior orbital adipose tissues obtained during decompression surgeries for GO and blepharoplasty for non-GO subjects (GOFs and N-OFs, respectively). Under standard 2D culture conditions, N-OFs and G-OFs displayed similar shape and proliferation as assessed by staining for Ki-67, a marker of cell proliferation (FIG. 1a). OFs from each group were equally positive for Thy-1 and α-SMA, markers representing lipogenic and contractile phenotypes of orbital fibroblasts (14) (FIG. 1a). Consistent with equal expression of these markers, a similar number of OFs (˜20% total) from each group differentiated into lipid-laden adipocytes in response to an adipogenic cocktail (containing insulin, troglitazone, triiodothyronine, and dexamethasone) (FIG. 1a).

To reproduce in vivo-like 3D tissue microenvironment, a high-throughput hanging droplet culture system was use (15). 20,000 OFs were used to generate each spheroidal organoid and the size, adipogenic potential, and tissue stiffness was assessed (FIG. 1b). Under both proliferating and adipogenic conditions, N-OFs and G-OFs formed uniformly shaped spheroidal organoids. Under adipogenic conditions, these OF-derived spheroids demonstrated adipogenic potentials as indicated by the presence of lipid-laden cells within a meshwork of ECM proteins, e.g, type VI collagen (FIG. 1c). 3D organoids derived from G-OFs had a larger cross-sectional area (CSA) than those from N-OFs after one day in culture (FIG. 1d). CSA of N-OF organoids declined over 6˜12 days of culture as spheroids became condensed (FIG. 1d). When adipogenic cocktail was added, N-OF CSA was maintained over 12 days in culture (FIG. 1d). This could be in part due to the emergence of BODIPY-positive adipocytes and ECM deposition within spheroids (FIG. 1c). GOF organoids also demonstrated progressive decline in CSA when cultured in standard medium; however, they were comparatively less responsive to the effect of adipogenic mix in maintaining spheroid size (FIG. 1c, d).

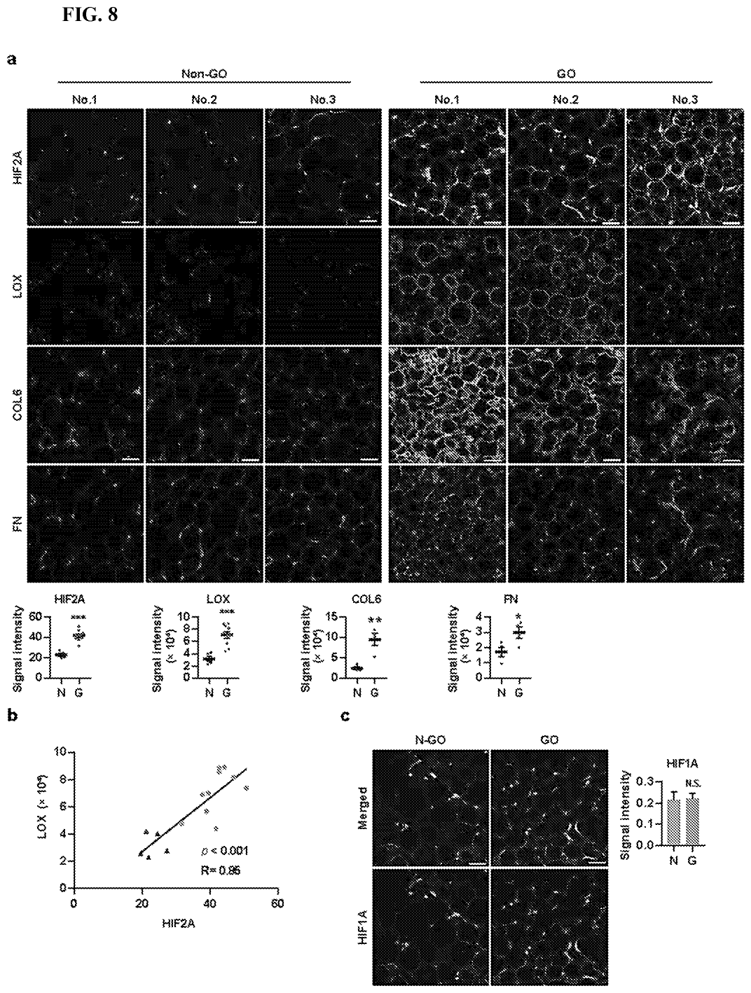

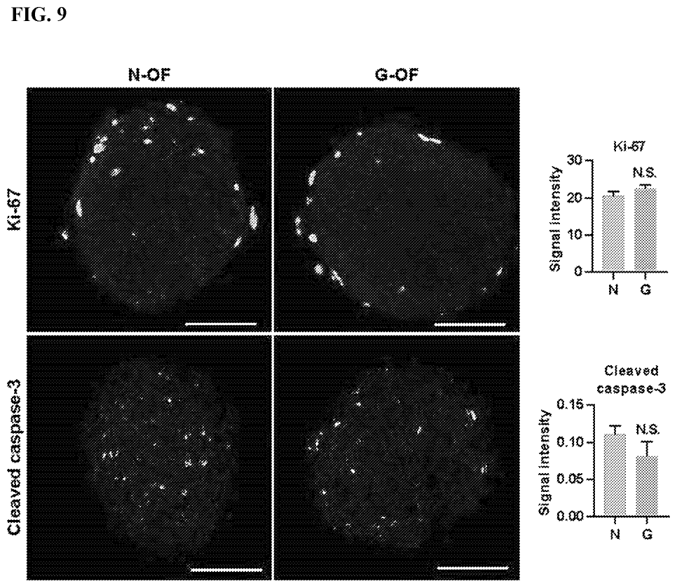

Next, the stiffness of 3D OF organoids was determined using compression-based force measurement. To do so, a microscale indentation technique (12) that provides real-time force-displacement measurements was used. Compression of organoids generated force-displacement in agreement with the previously-reported “viscoelastic model” (16, 17). 3D N-OF organoids cultured for 12 days required higher force and energy to achieve 50% strain compared with organoids cultured for 6 days, suggesting a time-dependent increase in tissue stiffness in culture (FIG. 1e). 3D G-OF organoids showed significantly higher tissue stiffness when compared to N-OF organoids at both time points (FIG. 1e). Notably, this difference was not due to altered cell proliferation or apoptosis between groups as assessed by staining for Ki-67 and cleaved caspace-3 (FIG. 9). Adipogenic stimulation markedly augmented tissue stiffness of both N-OF and G-OF organoids: however, the stiffness of G-OF organoids remained nearly twice as high as that of N-OF organoids (FIG. 1f). Together, the data demonstrate that G-OF organoids demonstrate greater tissue stiffness than N-OF organoids under both proliferating and adipogenic conditions.

ECM Accumulation within OF Organoids Determines Tissue Stiffness

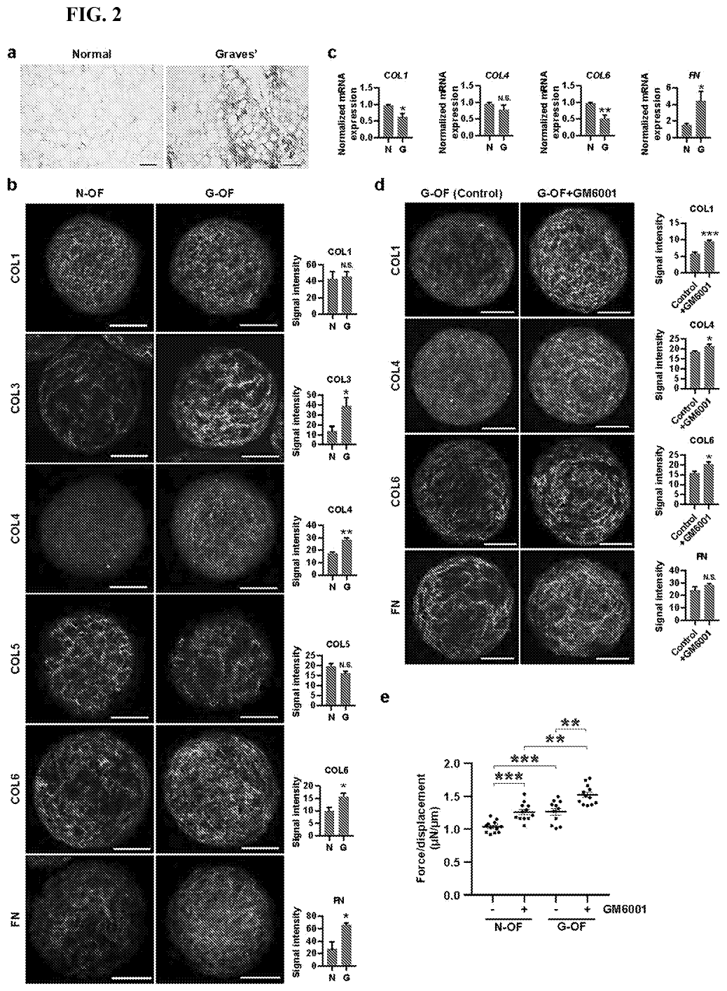

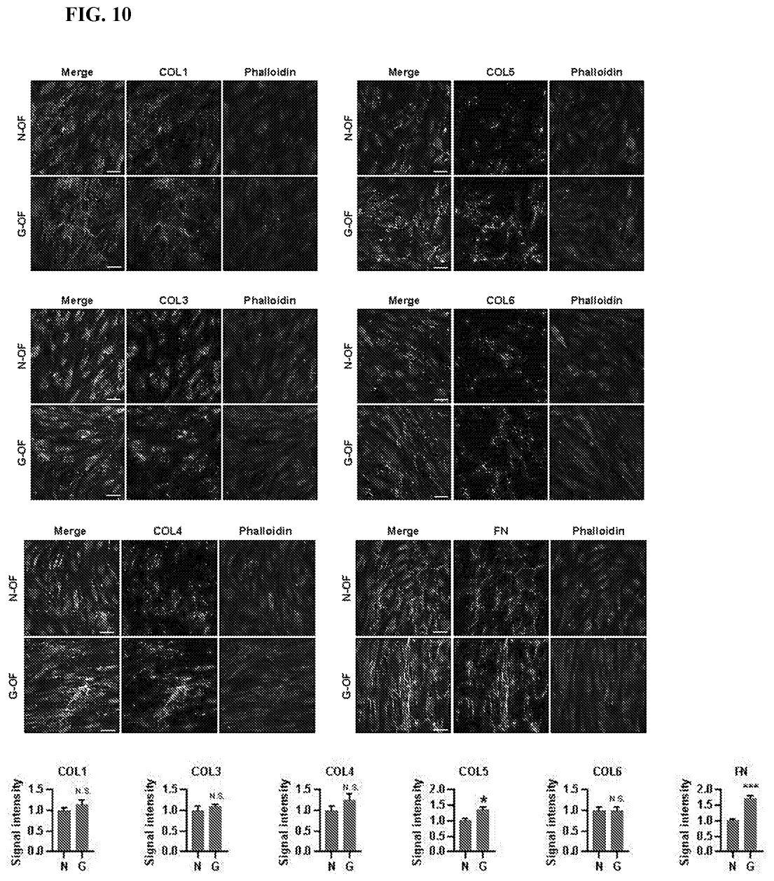

Orbital adipose tissues from Graves' and non-Graves' patients was stained with the collagen binding dye, picrosirius red. Graves' orbital adipose tissue had higher overall collagen content relative to non-Graves' control (FIG. 2a). When quantitative immunohistochemistry for collagen subtypes was performed on G-OFs and N-OFs grown in 2D culture, no difference in type I, III, IV, or VI collagen was observed, but significant increase in type V collagen and fibronectin (FN) in G-OF group was observed (FIG. 10). Assembling the same cells into 3D organoids accelerated the accumulation of type III, IV, and VI collagens, as well as FN in G-OF organoids but not in N-OF group (FIG. 2b). While FN gene expression was higher in G-OF than N-OF organoids, transcripts encoding type I, IV, and VI collagen were unchanged or rather reduced in G-OF organoids (FIG. 2c). This indicates that increased ECM deposition in G-OF organoids is regulated post-transcriptionally, e.g., by a post-translational mechanism to promote ECM fibrillogenesis (18-20). To test whether posttranslational ECM remodeling modifies ECM deposition and tissue rigidity, the role of proteinase-dependent ECM turnover in the regulation of ECM deposition and tissue stiffness was assayed. Matrix metalloproteinase (MMP) family members play a dominant role in collagen remodeling (21). When N-OF and G-OF spheroids were treated with pan-MMP inhibitor, GM6001, which would delay MMP-dependent collagen turnover, significant accumulation of type I, IV, and VI collagens was observed (FIG. 2d) in parallel with increased tissue stiffness (FIG. 2e). These results indicate that MMP-modifiable ECM deposition plays a key role in determining the tissue stiffness of OF-organoids.

### TSHR Activation Promotes the Fibrosis and Stiffness of Graves' OF Organoids

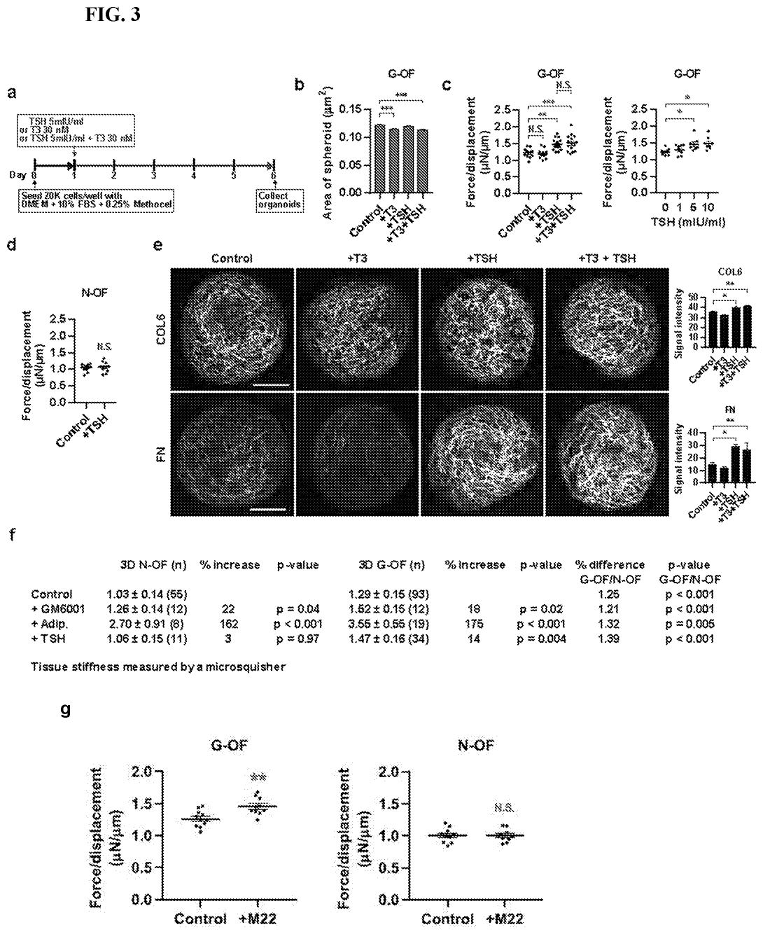

TSHR is expressed in Graves' OFs and considered to play a pathological role in GO as well as in hypothyroidism-associated ophthalmopathy (22, 23). Since OFs express both thyroid hormone receptor (TR) and TSHR, the effects of triiodothyronine (T3), thyroid stimulating hormone (TSH), and both together on the tissue stiffness and ECM deposition of OF organoids was assayed. Organoids were cultured in the presence of T3 (30 nM), TSH (5 mIU/ml), or combination (T3+TSH) over a 6-day time course (FIG. 3a). While TSH did not impact the size of G-OF organoids, T3 and T3+TSH yielded a significant reduction in CSA (FIG. 3b). By contrast, TSH increased the stiffness of 3D Graves' OF organoids but not T3 (FIG. 3c). The response of G-OF organoids to TSH was dose-dependent (FIG. 3c). Nonetheless, TSH had no impact on the stiffness of N-OF organoids (FIG. 3d), indicating genetic or epigenetic changes in G-OFs potentially mediate the differential response of G-OFs to TSH. T3 and TSH dependent ECM deposition in G-OF organoids was next evaluated. While T3 had no discemable impact, TSH and TSH+T3 induced robust accumulation of both type VI collagen and FN (FIG. 3e). These results together indicate that while the tissue stiffness of 3D N-OF and G-OF organoids is regulated by MMP and adipogenic cues, G-OF organoids display higher baseline tissue stiffness, which is further augmented by adipogenic stimuli and TSHR activation (FIG. 3f). Supporting the role of TSHR activation in increasing TAO orbital tissue stiffness in the pathological milieu of Grave's disease TSHR-activating immunoglobulin (M22) increased the stiffness of G-OF organoids but not N-OF organoids (FIG. 3g).

### 3D Specific LOX Activity is Responsible for the Mechanical Stiffness of G-OF Organoids.

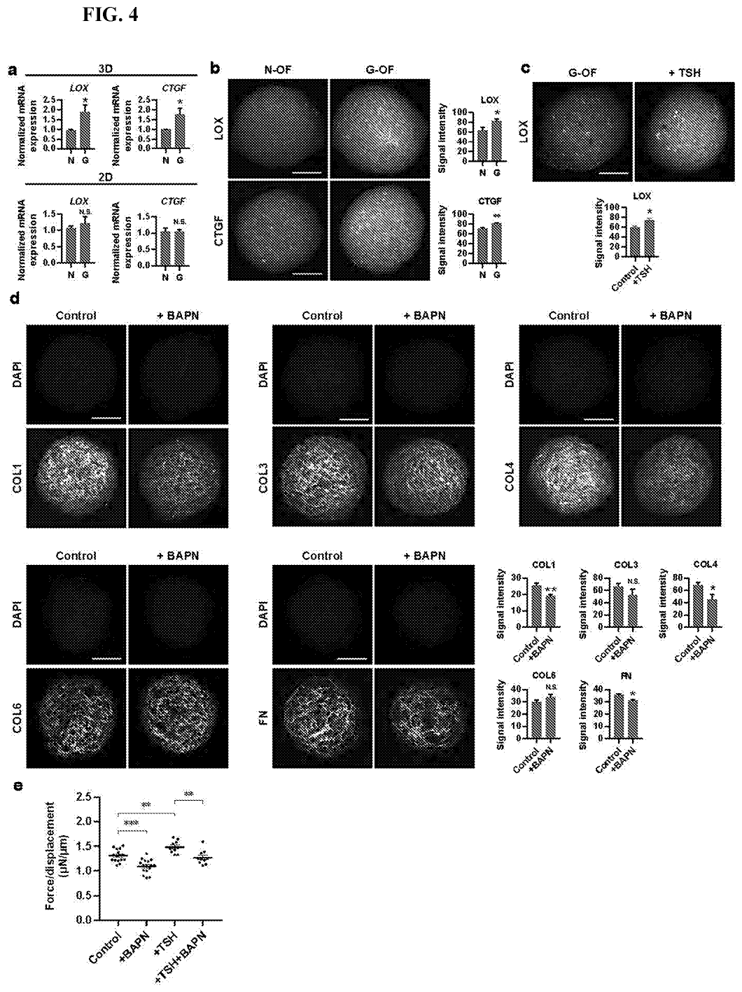

Since a concomitantly elevated collagen gene expression was not observed in parallel with increased ECM deposition in G-OF-spheroids (FIG. 2b), it was contemplated that non-transcriptional mechanisms of collagen deposition may underlie excess ECM accumulation seen in G-OF organoids. To test this, the gene expression of lysyl oxidase (LOX), a key mediator of collagen cross-linking, and of connective tissue growth factor (CTGF), a matricellular protein that promotes fibrillogenesis (24, 25) were measured. Basal levels of LOX and CTGF expression were significantly higher in G-OF organoids relative to N-OF organoids (FIG. 4a). However, no difference in expression of these genes was observed when cells were cultured in 2D condition (FIG. 4a). Congruent with these findings, quantitative immunocytochemistry demonstrated increased LOX and CTGF protein content in G-OF versus N-OF organoids (FIG. 4b). Furthermore, LOX protein was further increased in G-OF organoids stimulated by TSH (FIG. 4c). Taken together, these data identify significant intrinsic increases in LOX and CTGF expression in organoids derived from patients with GO, which may contribute to the increased ECM deposition and tissue stiffness of G-OF organoids.

To assess the role of LOX in 3D G-OF-specific ECM deposition and tissue stiffness, the effect of β-aminopropionitrile (BAPN), an irreversible inhibitor of LOX (26), on ECM deposition and organoid stiffness was assayed. BAPN treatment effectively suppressed the deposition of collagens type I and IV, FN, but not collagen type III and VI (FIG. 4d). Suppressed collagen crosslinking and fibronectin deposition led to the reduction of tissue stiffness as measured by micro-indentation tests (FIG. 4e). These results point to a major role for collagen crosslinking activity, which is mainly mediated by LOX, in increasing tissue stiffness of G-OF organoids.

### Proinflammatory Characteristics of 3D G-OF Organoids

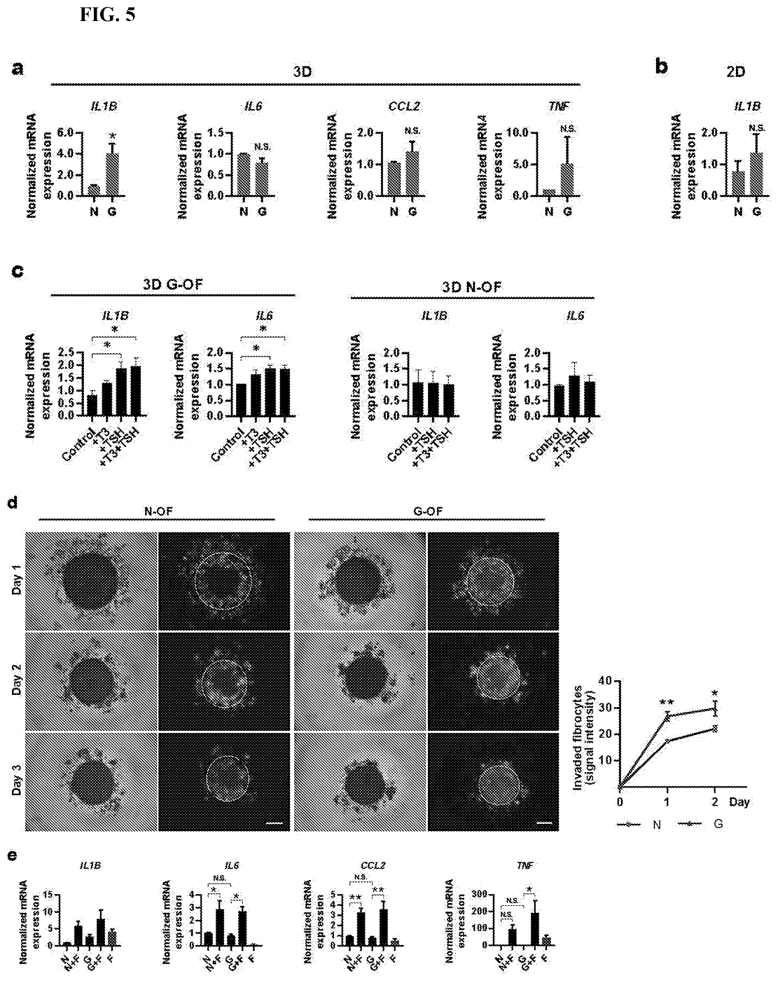

In vivo, Graves' orbital tissues demonstrate increased inflammation and fibrosis in GO disease progression (27). To determine whether this phenotype would be recapitulated in the system described herein, expression of known GO pro-inflammatory genes (2) was quantified in organoids derived from GOFs and N-OFs. the expression of the gene encoding interleukin 1 beta (IL1B) was increased in G-OF organoids, whereas genes encoding interleukin 6 (IL6), monocyte chemotactic protein 1 (CCL2), and tumor necrosis factor (TNF) unchanged between groups (FIG. 5a). No difference was observed in IL1B expression between G-OFs and N-OFs when grown in 2D culture (FIG. 5b). Moreover, G-OF organoids responded to TSH by increasing the levels of IL1B and IL6; however, no synergistic effects were observed between T3 and TSH in regulating the expression of IL1B and IL6 (FIG. 5c). The effect of TSH on IL1B and IL6 expression was specific for G-OF organoids and not observed with N-OF organoids.

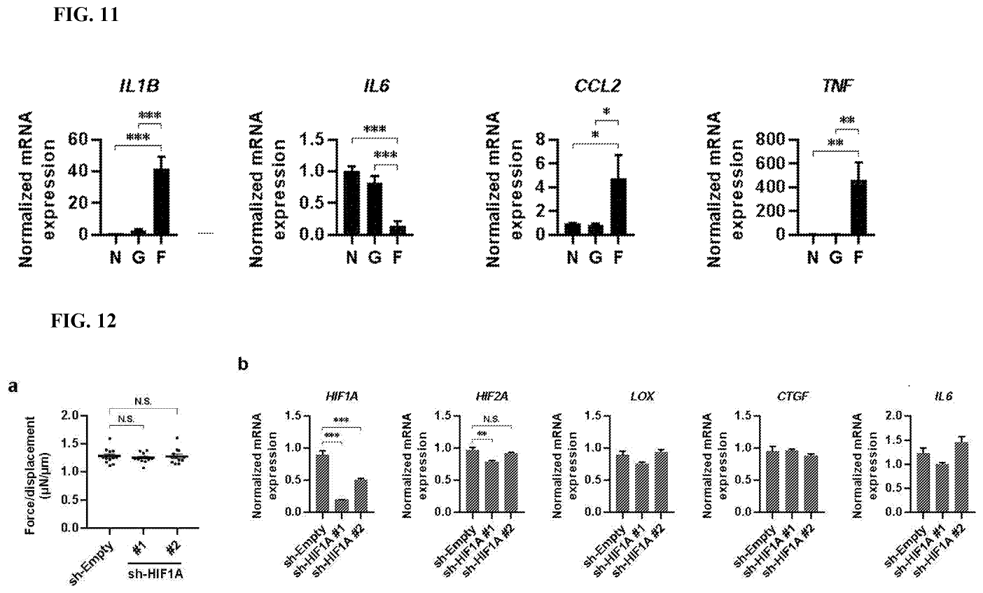

Infiltration of lymphocytes and fibrocytes/macrophages is another component of pro-inflammatory phenotype characterizing GO (2). 3D organoid culture is advantageous for recapitulating such heterotopic cell invasion in a tissue-like context. Human fibrocytes were differentiated in vitro from human peripheral mononuclear blood cells (28) and labeled with GFP using adenoviral gene transfer. When GFP-labeled fibrocytes were added to N—OF and G-OF 3D organoids in a hanging droplet culture, fibrocytes preferentially infiltrated into G-OF organoids in a time-dependent manner (FIG. 5d). In this model, the interplay between fibrocytes and OFs in transcriptomic regulation of 3D organoids was observed. Fibrocytes express higher levels of IL1B, CCL2, and TNF than OFs (FIG. 11). When added to N—OF or G-OF organoids at the ratio of 1:10 cell number ratio, fibrocytes induced an increase in the expression of IL6 and CCL2 in both N-OF and G-OF organoids, and of TNF only in G-OF organoids (FIG. 5e). IL1B showed only an additive increase conferred by fibrocytes. These data indicate intrinsic properties of G-OFs that promote fibrocyte migration, and synergistic induction of inflammatory cytokine expression through the interaction between OFs and fibrocytes.

### Hypoxia Inducible Factor 2A Drives Fibrosis and Tissue Stiffness in Graves' Orbitopathy

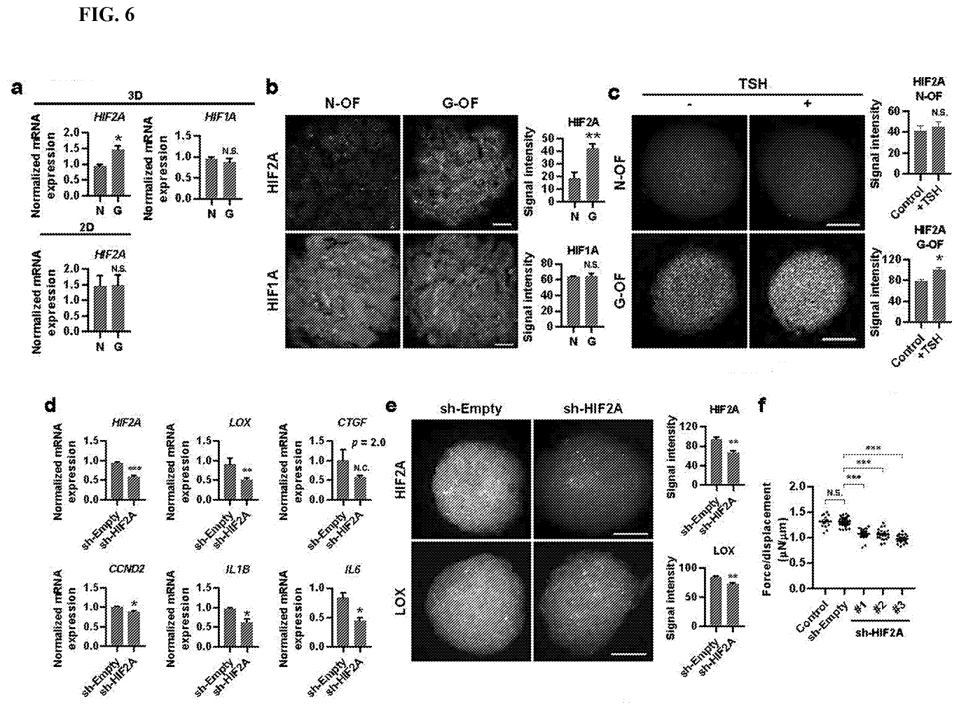

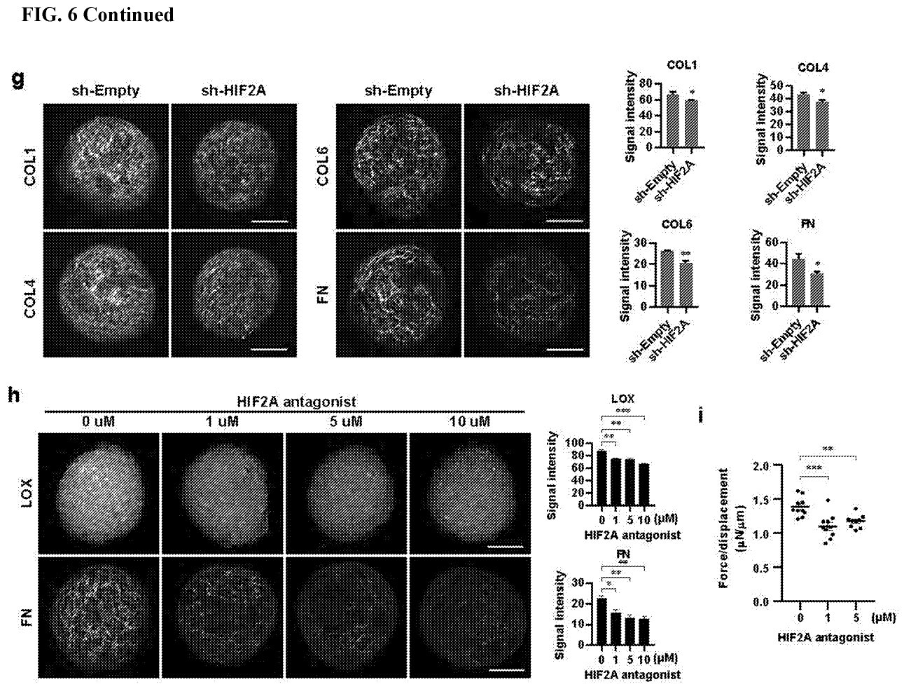

Hypoxia inducible factors, (HIF1A and HIF2A), promote inflammation and fibrosis (29, 30). HIF1A and HIF2A are also known as upstream regulators of CTGF and LOX expression (29, 31, 32). It was hypothesized that HIF family members are involved in the upregulated CTGF and LOX expression to drive fibrosis and inflammation. The expression of HIF1A and HIF2A was quantified in N-OF and G-OF organoids and a higher expression of HIF2A but not HIF1A was observed in G-OF organoids (FIG. 6a). In parallel, HIF2A but not HiF1A protein content was significantly higher in GOF organoids than N-OF organoids (FIG. 6b). HIF2A content in G-OF but not N-OF organoids increased upon TSHR activation by TSH (FIG. 6c). It was contemplated that increased HIF2A might underlie the increased collagen fibrillogenesis and tissue stiffness of G-OF organoids through the induction of LOX and CTGF. To test this, the impact of shRNA-mediated HIF2A knockdown on ECM remodeling and tissue stiffness in G-OF organoids was evaluated. HIF2A suppression using three independent lentiviral shRNA clones (average ˜40% transcript reduction) reduced the expression of known HIF target genes, LOX, IL1B, IL6, and CCND2(cyclin D2) (32, 33) (FIG. 6d). In parallel, reduction in protein levels of HIF2A and LOX was observed (FIG. 6e). Consistent with the hypothesis, HIF2A knockdown significantly reduced tissue stiffness of G-OF organoids (FIG. 6f), and levels of collagen type I, IV, VI, and FN proteins (FIG. 6g). Treatment of G-OF organoids with a HIF2A antagonist (C₁₂H₆ClFN₄O₃) (34) showed a similar effect, reducing protein levels of LOX and FN, in a dose-dependent manner (FIG. 6h) and then normalized the tissue stiffness of G-OF organoids (FIG. 6i). The effects of shRNA-mediated inhibition of HIF1A on tissue stiffness of G-OF organoids was assayed. Even though HIF1A transcript was suppressed by 78% and 43% with two unique shRNA clones, respectively, neither markedly impacted tissue stiffness and gene expression of LOX, CTGF or IL6 in G-OF organoids (FIG. 12). Taken together, these data demonstrate that HIF2A but not HIF1A is highly expressed in 3D organoids derived from patients with GO, where HIF2A upregulates gene and protein expression of LOX, CTGF, multiple collagen subtypes. FN, and IL6, to drive tissue stiffness and inflammation.

### HIF2A Activation is Sufficient to Induce Tissue Fibrosis and Rigidity

To further investigate the causal relationship between HIF2A activity and tissue stiffness, a mouse model wherein a HIF2A mutant with proline to alanine substitution (HIF2dPA), resistant to von Hippel-Lindau (VHL)-dependent degradation, is induced by Cre recombinase (35) was used. Primary OFs were isolated from orbital adipose tissues of ROSA26-HIF2dPA mice (35), treated in vitro with adenoviral Cre, and 3D organoids were generated from these cells (FIG. 7a). It was found that HIF2A transcript and protein were significantly increased in organoids derived from OFs treated with Cre-expressing adenovirus versus those treated with control (GFP-expressing) adenovirus (FIG. 7b-c). In parallel, LOX expression was markedly induced in these organoids at gene and protein levels (FIG. 7b-c). In keeping with this finding, collagens type IV, VI and FN, were upregulated in organoids generated from adeno-Cre-treated mouse OFs harboring HIF2dPA mutant. In this mouse model, type I collagen expression was unchanged (FIG. 7d). Regardless, HIF2A-overexpressing mouse OF organoids became stiffer than controls (FIG. 7e). To determine whether HIF2A-dependent stiffness required LOX activity, these organoids were treated with a LOX inhibitor, BAPN (26). BAPN treatment completely ameliorated the increased tissue stiffness conferred by mutant HIF2A expression, indicating that HIF2A-dependent increase of tissue stiffness is mediated by lysyl oxidase activity (FIG. 7e). Furthermore, 111b expression was upregulated with mutant HIF2A expression, while 116 transcript level was unchanged in comparison to control mouse OF organoids (FIG. 7f). The findings indicate that the activation of HIF2A, thorough LOX induction, is sufficient to increase OF tissue stiffness, in a manner coupled with ECM deposition and inflammatory gene expression in 3D organoid models.

### Augmented Expression of HIF2A and LOX in Graves' Orbitopathy

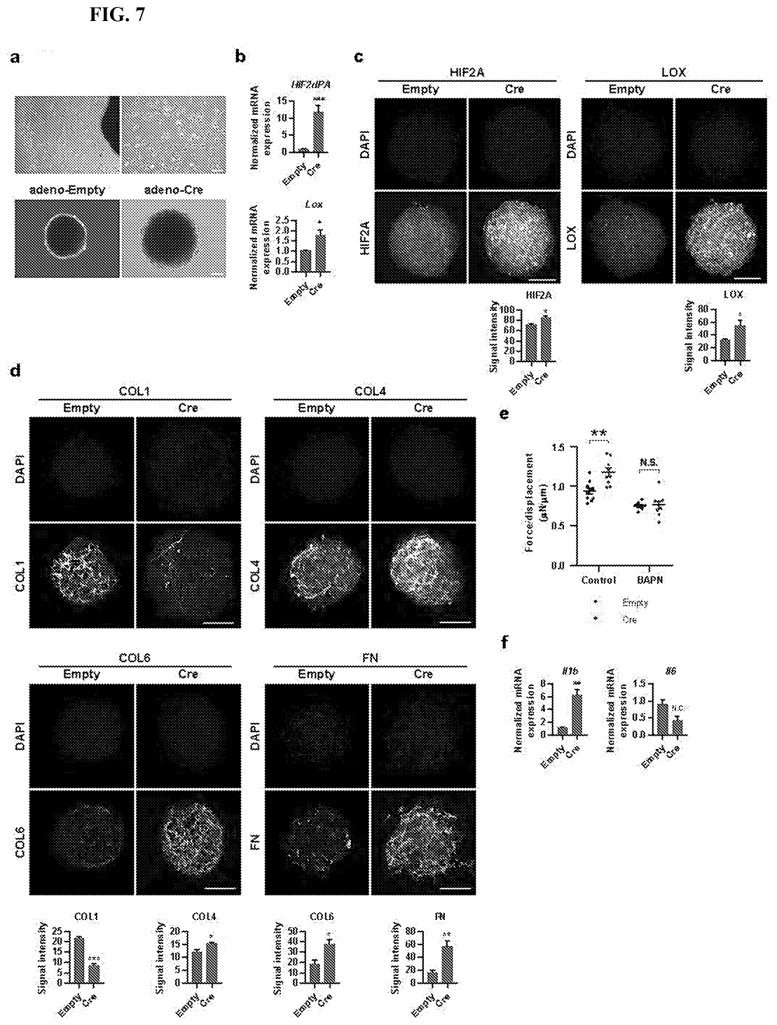

To validate the ex vivo findings, quantitative immunohistochemistry was used to evaluate HIF2A, LOX, collagen species, and FN in orbital adipose tissues isolated from human subjects with and without GO. HIF2A, LOX, type VI collagen and FN staining were low in normal orbital adipose tissues but substantially higher in those from patients with GO (FIG. 8a). A positive correlation between signal intensity of HIF2A and LOX was detected in GO tissues (FIG. 8b). HIF1A staining was similar in GO and non-GO tissue (FIG. 8c).

### HIF2A Inhibitor is Effective in Reducing Tissue Stiffness Induced by TSHR Activation

To determine the efficacy of pharmacological inhibition of HIF2A in reducing the tissue stiffness of G-OF organoids in the hormonal milieu of Graves' disease, an HIF2A allosteric inhibitor (C₁₂H₆ClFN₄O₀₃) was tested in G-OF organoids stimulated by TSH or M22. In either TSH- or M22-stimulated G-OF organoids, pharmacological inhibition of HIF2A reversed the tissue stiffness caused by TSHR activation (FIG. 14).

TABLE 1

| | | SEQ | | SEQ | | SEQ |
|---|---|---|---|---|---|---|
| | | ID | | ID | TagMan probe | ID |
| Gene | Forward primer (5′ to 3′) | NO: | Reverse primer (5′ to 3′) | NO: | (5′ to 3′) | NO: |
| HIF2A | CTTTGCGAGCATCCGGTA | 6 | AGCCTATGAATTCTACCA | 7 | | |
| | | | TGCG | | | |
| HIF2dPA | CGGAGGTGTTCTATGAGCTG | 8 | AGCTTGTGTGTTCGCAGG | 9 | | |
| | G | | AA | | | |
| HIF1A | CAACCCAGACATATCCACCT | 10 | CTCTGATCATCTGACCAA | 11 | | |
| | C | | AACTCA | | | |
| LOX | TTCCCACTTCAGAACACCAG | 12 | ACATTCGCTACACAGGAC | 13 | | |
| | | | ATC | | | |
| CTGF | CACCCGGGTTACCAATGACA | 14 | GGATGCACTTTTTGCCCTT | 15 | | |
| | | | CTTA | | | |
| COL1A1 | TTCTGTACGCAGGTGATTGG | 16 | GACATGTTCAGCTTTGTG | 17 | | |
| | | | GAC | | | |
| COL4A1 | TGAGTCAGGCTTCATTATGTT | 18 | AGAGAGGAGCGAGATGT | 19 | | |
| | CT | | TCA | | | |
| COL6A1 | GTGAGGCCTTGGATGATCTC | 20 | CCTCGTGGACAAAGTCAA | 21 | | |
| | | | GT | | | |
| FN | TTTGACCCCTACACAGTTTCC | 22 | TGACCACTTCCAAAGCCT | 23 | | |
| | | | AAG | | | |
| IL1B | GAACAAGTCATCCTCATTGC | 24 | CAGCCAATCTTCATTGCT | 25 | | |
| | C | | CAAG | | | |
| IL6 | TTCTGTGCCTGCAGCTTC | 26 | GCAGATGAGTACAAAAG | 27 | /FAM/CAACCA | 28 |
| | | | TCCTGA | | CAA/ZEN/ATG | |
| | | | | | CCAGCCTGCT | |
| | | | | | /IABkFQ/ | |
| TNF | TCAGCTTGAGGGTTTGCTAC | 29 | TGCACTTTGGAGTGATCG | 30 | | |
| | | | G | | | |
| CCL2 | GCCTCTGCACTGAGATCTTC | 31 | AGCAGCACCTTCATTCC | 32 | | |
| CCND2 | CCTCCAAACTCAAAGAGACC | 33 | TTCCACTTCAACTTCCCCA | 34 | | |
| | AG | | G | | | |
| 36B4 | TGTCTGCTCCCACAATGAAA | 35 | TCGTCTTT | 36 | /FAM/CCCTGT | 37 |
| | C | | AAACCCTGCGTG | | CTT/ZEN/CCC | |
| | | | | | TGGGCATCAC | |
| | | | | | /IABkFQ/ | |
| Mouse | | | | | | |
| Lox | CAAGGGACATCGGACTTCTT | 38 | TGGCATCAAGCAGGTCAT | 39 | | |
| | AC | | AG | | | |
| Ctgf | GCTGACCTGGAGGAAAACA | 40 | CCAGAAAGCTCAAACTTG | 41 | | |
| | TTA | | ACAG | | | |
| Col1a1 | CA | 42 | CGCAAAGAGTCTACATGT | 43 | /FAM/CCGGAG | 44 |
| | TTGTGTATGCAGCTGACTT | | CTAGG | | GTC/ZEN/CAC | |
| | C | | | | AAAGCTGAAC | |
| | | | | | A/IABkFQ/ | |
| Col4a1 | AATCCAATGACACCTTGCAA | 45 | TCTGGCTGTGGAAAATGT | 46 | /FAM/TCTTTCT | 47 |
| | C | | GA | | CC/ZEN/CTTTT | |
| | | | | | GTCCCTTCAC | |
| | | | | | GC/IABkFQ/ | |
| Col6a1 | AAGTTCTGTAGGCCAATGCT | 48 | CCAGATGAGTGTGAGATC | 49 | | |
| | C | | CTG | | | |
| II1b | TGCCACCTTTTGACAGTGAT | 50 | ATGTGCTGCTGCGAGATT | 51 | | |
| | G | | TG | | | |
| II6 | TCCTTAGCCACTCCTTCTGT | 52 | AGCCAGAGTCCTTCAGA | 53 | | |
| | | | GA | | | |
| 36b4 | TTATAACCCTGAAGTGCTCG | 54 | CGCTTGTACCCA | 55 | /FAM/AGGCCC | 56 |
| | AC | | TTGATGATG | | TGC/ZEN/ACT | |
| | | | | | CTCGCTT/IABkFQ/ | |

### Example 2

Experiments described above demonstrate that hypoxia-inducible factor (HIF2A)-dependent induction of lysyl oxidase (LOX) is responsible for the fibrotic tissue damage of orbital adipose tissue in thyroid-associated orbitopathy (TAO) (Hikage et al, Endocrinology 2019; 160(1):20-35).

Based on the finding, a high-throughput HIF2A-dependent LOX promoter activity assay adopting a method originally developed by Wang, Davis. and Yarchoan was developed (Wang et al. Biochem Biophys Res Comm 2017; 49(2):480485) In this assay, 293T cells were transfected with LOX promoter (403 bp upstream of start codon) with luciferase reporter was transfected with human HIF2A expression vector. HIF2A transfection specifically increases LOX promoter activity, which was inhibited by HIF2A antagonist, PT2385. The safety profile of PT2385 in humans has been established (Courtney K D et al, J Clin Oncol 2018, PMID: 29257710) and it is currently in clinical trials for recurrent glioblastoma and renal cell carcinoma.

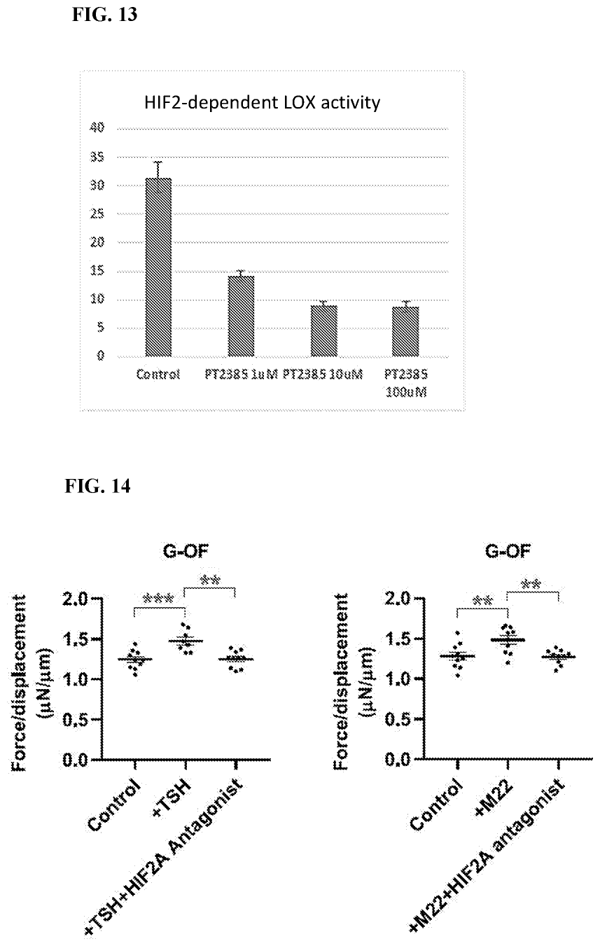

The results (FIG. 13) indicate that PT2385 is a potent HIF2A inhibitor that effectively blocks HIF2A-dependent induction of LOX, a key collagen crosslinking enzyme, essential in the pathogenic development of tissue fibrosis.

### REFERENCES

1. Bahn R S. Graves' ophthalmopathy. N Engl J Med. 2010; 362(8):726-738.
2. Smith T J, Hegedus L. Graves' Disease. N Engl J Med. 2016; 375(16):1552-1565.
3. Puente X S, Sanchez L M, Overall C M, Lopez-Otin C. Human and mouse proteases: a comparative genomic approach. Nat Rev Genet. 2003; 4(7):544-558.
4. Mestas J, Hughes C C W. Of Mice and Not Men: Differences between Mouse and Human Immunology. Jmmunol. 2004; 172(5):2731-2738.
5. Zhao S X, Tsui S, Cheung A. Douglas R S, Smith T J, Banga J P. Orbital fibrosis in a mouse model of Graves' disease induced by genetic immunization of thyrotropin receptor cDNA. J Endocrinol. 2011; 210(3):369-377.
6. Moshkelgosha S, So P W, Deasy N, Diaz-Cano S, Banga J P. Cutting edge: retrobulbar inflammation, adipogenesis, and acute orbital congestion in a preclinical female mouse model of Graves' orbitopathy induced by thyrotropin receptor plasmid-in vivo electroporation. Endocrinology. 2013; 154(9):3008-3015.
7. Many M C, Costagliola S, Detrait M, Denef F, Vassart G Ludgate M C. Development of an animal model of autoimmune thyroid eye disease. J Immunol. 1999162(8):4966-4974.
8. Baker G, Mazziotti G, von Ruhland C, Ludgate M. Reevaluating thyrotropin receptorinduced mouse models of graves' disease and ophthalmopathy. Endocrinology. 2005; 146(2):835-844.
9. Gower N J D, Barry R J, Edmunds M R, Titcomb L C, Denniston A K. Drug discovery in ophthalmology: past success, present challenges, and future opportunities. BMC Ophthalmol. 2016; 16(1):11.
10. Huh D, Hamilton G A, Ingber D E. From 3D cell culture to organs-on-chips. Trends Cell Biol. 2011; 21(12):745-754.
11. Wang F, et al. Reciprocal interactions between beta 1-integrin and epidermal growth factor receptor in three-dimensional basement membrane breast cultures: A different perspective in epithelial biology. Proc Natl Acad Sci USA. 1998; 95(25):14821-14826.
12. Yoo L. et al. Characterization of Ocular Tissues Using Microindentation and Hertzian Viscoelastic Models. Invest Ophthalmol Vis Sci. 2011; 52(6):3475-3482.
13. Weetman A P. Graves' disease. NEngl J Med 2000343(17):1236-1248.
14. Koumas L, Smith T J, Feldon S, Blumberg N, Phipps R P. Thy-1 expression in human fibroblast subsets defines myofibroblastic or lipofibroblastic phenotypes. AmJPathol. 2003; 163(4):1291-1300.
15. Moraes C, Labuz J M, Leung B M. Inoue M. Chun T H. Takayama S. On being the right size: scaling effects in designing a human-on-a-chip. Integr Biol (Camb). 2013; 5(9):1149-1161.
16. Kumar S, et al. Viscoelastic retraction of single living stress fibers and its impact on cell shape, cytoskeletal organization, and extracellular matrix mechanics. Biophys J. 2006; 90(10):3762-3773.
17. Forgacs G, Foty R A, Shafrir Y, Steinberg M S. Viscoelastic properties of living embryonic tissues: a quantitative study. Biophys J. 1998; 74(5):2227-2234.
18. Kadler K E, Hill A. Canty-Laird E G. Collagen fibrillogenesis: fibronectin, integrins, and minor collagens as organizers and nucleators. Curr Opin Cell Biol. 2008; 20(5-24):495-501.
19. McDonald J A, Kelley D G, Broekelmann T J. Role of fibronectin in collagen deposition: Fab′ to the gelatin-binding domain of fibronectin inhibits both fibronectin and collagen organization in fibroblast extracellular matrix. J Cell Biol. 1982; 92(2):485-492.
20. Herchenhan A. et al. Lysyl Oxidase Activity Is Required for Ordered Collagen Fibrillogenesis by Tendon Cells. J Biol Chem. 2015; 290(26):16440-16450.
21. Kessenbrock K. Plaks V, Werb Z. Matrix metalloproteinases: regulators of the tumor microenvironment. Cell. 2010; 141(1):52-67.
22. Mengistu M, et al. TSH receptor gene expression in retroocular fibroblasts. JEndocrinol Invest. 1994; 17(6):437-441.
23. Bell A, Gagnon A, Grunder L, Parikh S J, Smith T J, Sorisky A. Functional TSH receptor in human abdominal preadipocytes and orbital fibroblasts. Am J Physiol Cell Physiol. 2000; 279(2):C335-340.
24. Frazier K, Williams S, Kothapalli D. Klapper H, Grotendorst G R. Stimulation of fibroblast cell growth, matrix production, and granulation tissue formation by connective tissue growth factor. J Invest Dermatol. 1996107(3):404-411.
25. Quan T, Shao Y, He T, Voorhees J J, Fisher G J. Reduced expression of connective tissue growth factor (CTGF/CCN2) mediates collagen loss in chronologically aged human skin. J Invest Dermatol. 2010; 130(2):415-424.
26. Narayanan A S, Siegel R C, Martin G R. On the inhibition of lysyl oxidase by -aminopropionitrile. Biochem Biophys Res Commun. 1972; 46(2):745-751.
27. Smith T J. TSH-receptor-expressing fibrocytes and thyroid-associated ophthalmopathy. Nat Rev Endocrinol. 2015; 11(3):171-181.
28. Bucala R, Spiegel L A, Chesney J, Hogan M, Cerami A. Circulating fibrocytes define a new leukocyte subpopulation that mediates tissue repair. Mol Med. 1994; 1(1):71-81.
29. Halberg N, et al. Hypoxia-inducible factor 1alpha induces fibrosis and insulin resistance in white adipose tissue. Mol Cell Biol. 2009; 29(16):4467-4483.
30. Qu A J, et al. Hypoxia-Inducible Transcription Factor 2 alpha Promotes Steatohepatitis Through Augmenting Lipid Accumulation, Inflammation, and Fibrosis. Hepatology. 2011; 54(2):472-483.
31. Higgins D F, Biju M P, Akai Y, Wutz A, Johnson R S, Haase V H. Hypoxic induction of Ctgf is directly mediated by Hif-1. Am J Physiol Renal Physiol. 2004; 287(6):F1223-1232.
32. Chen W, et al. Targeting renal cell carcinoma with a HIF-2 antagonist. Nature. 2016; 539(7627):112-117.
33. Gordan J D, Bertout J A, Hu C J, Diehl J A, Simon M C. HIF-2alpha promotes hypoxic cell proliferation by enhancing c-myc transcriptional activity. Cancer Cell. 2007; 11(4):335-347.
34. Scheuermann T H, et al. Allosteric inhibition of hypoxia inducible factor-2 with small molecules. Nat Chem Biol. 2013; 9(4):271-276.
35. Kim W Y, et al. Failure to prolyl hydroxylate hypoxia-inducible factor alpha phenocopies VHL inactivation in vivo. EMBO J. 2006; 25(19):4650-4662.
36. Thoma C R, Zimmermann M. Agarkova I, Kelm J M, Krek W. 3D cell culture systems modeling tumor growth determinants in cancer target discovery. Adv Drug Deliv Rev. 2014; 69-70:29-41.
37. Chun T H. Hotary K B, Sabeh F, Saltiel A R, Allen E D, Weiss S J. A pericellular collagenase directs the 3-dimensional development of white adipose tissue. Cell. 2006; 125(3):577-591.
38. Wilson W B, Manke W F. Orbital decompression in Graves' disease. The predictability of reduction of proptosis. Arch Ophthalmol. 1991; 109(3):343-345.
39. Tung Y C, Hsiao A Y, Allen S G, Torisawa Y S, Ho M, Takayama S. High-throughput 3D spheroid culture and drug testing using a 384 hanging drop array. Analyst. 2011; 136(3):473-478.
40. Wang W, et al. 3D spheroid culture system on micropattemed substrates for improved differentiation efficiency of multipotent mesenchymal stem cells. Biomaterials. 2009; 30(14):2705-2715.
41. Discher D E, Smith L, Cho S, Colasurdo M, Garcia A J, Safran S. Matrix Mechanosensing: From Scaling Concepts in ‘Omics Data to Mechanisms in the Nucleus, Regeneration, and Cancer. Annu Rev Biophys. 2017; 46(1):295-315.
42. Ralphs J R, Waggett A D, Benjamin M. Actin stress fibres and cell-cell adhesion molecules in tendons: organisation in vivo and response to mechanical loading of tendon cells in vitro. Matrix Biol. 2002; 21(1):67-74.
43. Wang V. Davis D A, Haque M, Huang L E, Yarchoan R. Differential gene up-regulation by hypoxia-inducible factor-1alpha and hypoxia-inducible factor-2alpha in HEK293T cells. Cancer Res. 2005; 65(8):3299-3306.
44. Wang V, Davis D A, Yarchoan R. Identification of functional hypoxia inducible factor response elements in the human lysyl oxidase gene promoter. Biochem Biophys Res Commun. 2017; 490(2):480-485.
45. Erler J T, et al. Lysyl oxidase is essential for hypoxia-induced metastasis. Nature. 2006; 440(7088):1222-1226.
46. Jensen J A, Goodson W H, Hopf H W, Hunt T K. Cigarette smoking decreases tissue oxygen. Arch Surg. 1991; 126(9):1131-1134.
47. Aragona M, et al. A Mechanical Checkpoint Controls Multicellular Growth through YAP/TAZ Regulation by Actin-Processing Factors. Cell. 2013; 154(5):1047-1059.
48. Preisser F, Giehl K, Rehm M, Goppelt-Struebe M. Inhibitors of oxygen sensing prolyl hydroxylases regulate nuclear localization of the transcription factors Smad2 and YAP/TAZ involved in CTGF synthesis. Biochim Biophys Acta. 2016; 1863(8):2027-2036.
49. Smith T J, Sempowski G D, Wang H S, Del Vecchio P J, Lippe S D, Phipps R P. Evidence for cellular heterogeneity in primary cultures of human orbital fibroblasts. J Clin Endocrinol Metab. 1995; 80(9):2620-2625.
50. Gortz G E, et al. Pathogenic Phenotype of Adipogenesis and Hyaluronan in Orbital Fibroblasts From Female Graves' Orbitopathy Mouse Model. Endocrinology. 2016; 157(10):3771-3778.
51. Bucala R, Spiegel L A, Chesney J, Hogan M, Cerami A. Circulating fibrocytes define a new leukocyte subpopulation that mediates tissue repair. Mol Med. 1994; 1(1):71-81.
52. Leung B M, Lesher-Perez S C, Matsuoka T, Moraes C, Takayama S. Media additives to promote spheroid circularity and compactness in hanging drop platform. Biomater Sci. 2015; 3(2):336-344.
53. Yu C, Kommuller A, Brown C. Hoare T, Flynn L E. Decellularized adipose tissue microcarriers as a dynamic culture platform for human adipose-derived stem/stromal cell expansion. Biomaterials. 2017; 120:66-80.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A method of preventing or treating thyroid eye disease in a subject, comprising:
inhibiting at least one activity or downregulating the expression of hypoxia-inducible factor alpha (HIF2A) or Lysyl Oxidase (LOX) in said subject under conditions such that said thyroid eye disease is treated or prevented.

2. The method of claim 1, wherein said inhibiting or downregulating the expression of HIF2A or LOX comprises the use of an agent selected from the group consisting of a nucleic acid, a small molecule, a peptide, a vector, and an antibody.

3. The method of claim 2, wherein said nucleic acid is selected from the group consisting of an siRNA, miRNA, an antisense nucleic acid, and an shRNA.

4. The method of claim 3, wherein said shRNA is on a lentiviral vector.

5. The method of claim 2, wherein said small molecule is selected from the group consisting of β-aminopropionitrile (BAPN), C₁₂H₆ClFN₄O₃, PT2385, and PT2399.

6. The method of claim 1, wherein said inhibiting at least one activity or downregulating the expression of HIF2A or LOX comprises inhibiting at least one activity or altering the expression of a HIF2A or LOX pathway member.

7. The method of claim 1, wherein said subject has Graves' disease.

8. A method of altering HIF2A or LOX activity in a cell, comprising:
inhibiting at least one activity or downregulating the expression of HIF2A or LOX in said cell.

9. The method of claim 8, wherein said inhibiting or downregulating the expression of HIF2A or LOX comprises the use of an agent selected from the group consisting of a nucleic acid, a small molecule, a peptide, a vector, and an antibody.

10. The method of claim 9, wherein said nucleic acid is selected from the group consisting of an siRNA, miRNA, an antisense nucleic acid, and an shRNA.

11. The method of claim 10, wherein said shRNA is on a lentiviral vector.

12. The method of claim 11, wherein said small molecule is selected from the group consisting of β-aminopropionitrile (BAPN), C₁₂H₆ClFN₄O₃, PT2385, and PT2399.

13. The method of claim 8, wherein said inhibiting at least one activity or downregulating the expression of HIF2A or LOX comprises inhibiting at least one activity or altering the expression of a HIF2A or LOX pathway member.

14. The method of claim 8, wherein said cell is in vitro, ex vivo, or in vivo.

15. The method of claim 14, wherein said cell is in a subject.

16. The method of claim 15, wherein said subject has Graves' disease.

17. The method of claim 15, wherein said subject has thyroid eye disease and said altering treats or prevents said thyroid eye disease.

18. An agent that inhibits at least one activity or downregulates the expression of HIF2A or LOX for use in treating or preventing thyroid eye disease in a subject.

19. The agent of claim 18, wherein agent is selected from the group consisting of a nucleic acid, a small molecule, a peptide, a vector, and an antibody.

20-21. (canceled)

22. The agent of claim 19, wherein said small molecule is selected from the group consisting of β-aminopropionitrile (BAPN), C₁₂H₆ClFN₄O₃, PT2385, and PT2399.

23-25. (canceled)


SEQUENCE LISTING (56 sequences)

SEQ ID NO: 1 - DNA, 18 bp, Artificial sequence
  Synthetic
gcattaaagcagcgtatc

SEQ ID NO: 2 - DNA, 57 bp, Artificial sequence
  Synthetic
ccgggcgcaaatgtacccaatgatactcgagtatcattgggtacatttgcgcttttt

SEQ ID NO: 3 - DNA, 57 bp, Artificial sequence
  Synthetic
ccggcagtacccagacggatttcaactcgagttgaaatccgtctgggtactgttttt

SEQ ID NO: 4 - DNA, 57 bp, Artificial sequence
  Synthetic
ccgggtgatgaaagaattaccgaatctcgagattcggtaattctttcatcacttttt

SEQ ID NO: 5 - DNA, 57 bp, Artificial sequence
  Synthetic
ccggtgctctttgtggttggatctactcgagtagatccaaccacaaagagcattttt

SEQ ID NO: 6 - DNA, 18 bp, Artificial sequence
  Synthetic
ctttgcgagcatccggta

SEQ ID NO: 7 - DNA, 22 bp, Artificial sequence
  Synthetic
agcctatgaattctaccatgcg

SEQ ID NO: 8 - DNA, 21 bp, Artificial sequence
  Synthetic
cggaggtgttctatgagctgg

SEQ ID NO: 9 - DNA, 20 bp, Artificial sequence
  Synthetic
agcttgtgtgttcgcaggaa

SEQ ID NO: 10 - DNA, 21 bp, Artificial sequence
  Synthetic
caacccagacatatccacctc

SEQ ID NO: 11 - DNA, 24 bp, Artificial sequence
  Synthetic
ctctgatcatctgaccaaaactca

SEQ ID NO: 12 - DNA, 20 bp, Artificial sequence
  Synthetic
ttcccacttcagaacaccag

SEQ ID NO: 13 - DNA, 21 bp, Artificial sequence
  Synthetic
acattcgctacacaggacatc

SEQ ID NO: 14 - DNA, 20 bp, Artificial sequence
  Synthetic
cacccgggttaccaatgaca

SEQ ID NO: 15 - DNA, 23 bp, Artificial sequence
  Synthetic
ggatgcactttttgcccttctta

SEQ ID NO: 16 - DNA, 20 bp, Artificial sequence
  Synthetic
ttctgtacgcaggtgattgg

SEQ ID NO: 17 - DNA, 21 bp, Artificial sequence
  Synthetic
gacatgttcagctttgtggac

SEQ ID NO: 18 - DNA, 23 bp, Artificial sequence
  Synthetic
tgagtcaggcttcattatgttct

SEQ ID NO: 19 - DNA, 20 bp, Artificial sequence
  Synthetic
agagaggagcgagatgttca

SEQ ID NO: 20 - DNA, 20 bp, Artificial sequence
  Synthetic
gtgaggccttggatgatctc

SEQ ID NO: 21 - DNA, 20 bp, Artificial sequence
  Synthetic
cctcgtggacaaagtcaagt

SEQ ID NO: 22 - DNA, 21 bp, Artificial sequence
  Synthetic
tttgacccctacacagtttcc

SEQ ID NO: 23 - DNA, 21 bp, Artificial sequence
  Synthetic
tgaccacttccaaagcctaag

SEQ ID NO: 24 - DNA, 21 bp, Artificial sequence
  Synthetic
gaacaagtcatcctcattgcc

SEQ ID NO: 25 - DNA, 22 bp, Artificial sequence
  Synthetic
cagccaatcttcattgctcaag

SEQ ID NO: 26 - DNA, 18 bp, Artificial sequence
  Synthetic
ttctgtgcctgcagcttc

SEQ ID NO: 27 - DNA, 23 bp, Artificial sequence
  Synthetic
gcagatgagtacaaaagtcctga

SEQ ID NO: 28 - DNA, 22 bp, Artificial sequence
  Synthetic
caaccacaaatgccagcctgct

SEQ ID NO: 29 - DNA, 20 bp, Artificial sequence
  Synthetic
tcagcttgagggtttgctac

SEQ ID NO: 30 - DNA, 19 bp, Artificial sequence
  Synthetic
tgcactttggagtgatcgg

SEQ ID NO: 31 - DNA, 20 bp, Artificial sequence
  Synthetic
gcctctgcactgagatcttc

SEQ ID NO: 32 - DNA, 18 bp, Artificial sequence
  Synthetic
agcagccaccttcattcc

SEQ ID NO: 33 - DNA, 22 bp, Artificial sequence
  Synthetic
cctccaaactcaaagagaccag

SEQ ID NO: 34 - DNA, 20 bp, Artificial sequence
  Synthetic
ttccacttcaacttccccag

SEQ ID NO: 35 - DNA, 21 bp, Artificial sequence
  Synthetic
tgtctgctcccacaatgaaac

SEQ ID NO: 36 - DNA, 20 bp, Artificial sequence
  Synthetic
tcgtctttaaaccctgcgtg

SEQ ID NO: 37 - DNA, 22 bp, Artificial sequence
  Synthetic
ccctgtcttccctgggcatcac

SEQ ID NO: 38 - DNA, 22 bp, Artificial sequence
  Synthetic
caagggacatcggacttcttac

SEQ ID NO: 39 - DNA, 20 bp, Artificial sequence
  Synthetic
tggcatcaagcaggtcatag

SEQ ID NO: 40 - DNA, 22 bp, Artificial sequence
  Synthetic
gctgacctggaggaaaacatta

SEQ ID NO: 41 - DNA, 22 bp, Artificial sequence
  Synthetic
ccagaaagctcaaacttgacag

SEQ ID NO: 42 - DNA, 22 bp, Artificial sequence
  Synthetic
cattgtgtatgcagctgacttc

SEQ ID NO: 43 - DNA, 23 bp, Artificial sequence
  Synthetic
cgcaaagagtctacatgtctagg

SEQ ID NO: 44 - DNA, 23 bp, Artificial sequence
  Synthetic
ccggaggtccacaaagctgaaca

SEQ ID NO: 45 - DNA, 21 bp, Artificial sequence
  Synthetic
aatccaatgacaccttgcaac

SEQ ID NO: 46 - DNA, 20 bp, Artificial sequence
  Synthetic
tctggctgtggaaaatgtga

SEQ ID NO: 47 - DNA, 26 bp, Artificial sequence
  Synthetic
tctttctcccttttgtcccttcacgc

SEQ ID NO: 48 - DNA, 21 bp, Artificial sequence
  Synthetic
aagttctgtaggccaatgctc

SEQ ID NO: 49 - DNA, 21 bp, Artificial sequence
  Synthetic
ccagatgagtgtgagatcctg

SEQ ID NO: 50 - DNA, 21 bp, Artificial sequence
  Synthetic
tgccaccttttgacagtgatg

SEQ ID NO: 51 - DNA, 20 bp, Artificial sequence
  Synthetic
atgtgctgctgcgagatttg

SEQ ID NO: 52 - DNA, 20 bp, Artificial sequence
  Synthetic
tccttagccactccttctgt

SEQ ID NO: 53 - DNA, 19 bp, Artificial sequence
  Synthetic
agccagagtccttcagaga

SEQ ID NO: 54 - DNA, 22 bp, Artificial sequence
  Synthetic
ttataaccctgaagtgctcgac

SEQ ID NO: 55 - DNA, 21 bp, Artificial sequence
  Synthetic
cgcttgtacccattgatgatg

SEQ ID NO: 56 - DNA, 19 bp, Artificial sequence
  Synthetic
aggccctgcactctcgctt